(19) United States
(12) Patent Application Publication
(10) Pub. No.: US 2002/0141979 A1
(43) Pub. Date: Oct. 3, 2002

(54) Methods and compositions for STAT-1 conferred resistance to immunotoxicity

(76) Inventors: Guoxun Chen, Carrollton, TX (US); Christopher B. Newgard, Dallas, TX (US)

Correspondence Address: Steven L. Highlander, FULBRIGHT & JAWORSKI L.L.P., A REGISTERED LIMITED LIABILITY PARTNERSHIP, 600 CONGRESS AVENUE, SUITE 2400, AUSTIN, TX, 78701, US

(21) Appl. No.: 09/833,205

(22) Filed: Apr. 10, 2001

Related U.S. Application Data
(60) Provisional application No. 60/195,913, filed on Apr. 10, 2000.

(51) Int. Cl. A61K 48/00; C12N 5/08; C12N 15/86

(52) U.S. Cl. 424/93.21; 435/456; 435/366

(57) ABSTRACT

The present invention describes methods and compositions for preventing immuno-cytotoxicity of transplanted cells. The invention provides cells and expression constructs encoding STAT-1α polypeptides that prevent cytokine-mediated cytotoxicity. Thus, the invention provides treatment methods for preventing the rejection of transplanted cells by a host. In a particular embodiment, the invention provides for treatment of diabetes by transplanting into a subject insulinoma cells that secrete insulin in response to glucose and further express STAT-1α, thereby conferring resistance to inflammatory cytokines involved in the cell-rejection response.

The present application claims priority to co-pending U.S. Provisional Patent Application Serial No. 60/195,913 filed Apr. 10, 2000. The entire text of the above-referenced disclosure is specifically incorporated by reference herein without disclaimer.

The government may own rights in the present invention pursuant to grant number DK 55188 and T32 GM08203 from the National Institute of Health.

### BACKGROUND OF THE INVENTION

1. Field of the Invention

The present invention relates generally to the fields of immunology, molecular biology, diabetes and cell transplantation. More particularly, it concerns methods and compositions for preventing immunocytotoxicity of transplanted cells. In particular embodiments, the invention provides cells and expression constructs encoding STAT-1α polypeptides that prevent cytokine-mediated cytotoxicity against insulinoma cells.

2. Description of Related Art

While the stimulation of the immune system prevents and controls infection, it can have an adverse effect, as is the case with autoimmune diseases and with the rejection of cells and tissues during transplants. Cell-mediated immunity occurs when sensitized T cells directly damage cells or release cytokines that augment an inflammatory reaction. B cell production of antibodies that bind “self” antigens are referred to as autoantibodies. An association of an autoantibody with its antigen in intercellular fluid causes cell lysis and autoantibody-induced release of inflammatory mediators, induction of the complement pathway and activation of cytotoxic cells.

Insulin-dependent diabetes mellitus is an example of an autoimmune disease characterized by the immune responses described above, in which the β-cells of a diabetic subject's pancreas are selectively destroyed by their own immune system. Diabetes mellitus is a prevalent degenerative disease, characterized by insulin deficiency, which prevents normal regulation of blood glucose levels, a condition which leads to hyperglycemia and ketoacidosis. Insulin-dependent diabetes mellitus (IDDM, also known as Juvenile-onset, or Type I diabetes) represents approximately 15% of all human diabetes. IDDM is distinct from non-insulin dependent diabetes (NIDDM) in that only IDDM involves specific destruction of the insulin producing β-cells of the islets of Langerhans in the pancreas. The destruction of β-cells in IDDM appears to be a result of specific autoimmune attack in which the patient's own immune system recognizes and destroys the β-cells, but not the surrounding α (glucagon producing) or δ (somatostatin producing) cells that comprise the islet.

Current diabetes treatments are limited primarily to insulin injection. Early diabetes treatments utilized porcine or bovine insulin, and more recently human insulin injections. Insulin injections prevent severe hyperglycemia and ketoacidosis, but fail to prevent premature vascular deterioration, the leading cause of morbidity among diabetics. Even more recently, insulin delivery pumps have been used to deliver varying doses of insulin (Kaufman et al., 1999; Lorenz, 1999), but these devices also fail to prevent premature vascular deterioration in the diabetic subject.

Surgical transplantation of part or all of the pancreas is thought potentially to be a promising direction for diabetes treatment (Otonkoski et al., 1999; Steen, 1999). Significant effort has been devoted to the strategy of islet or pancreas fragment transplantation as a means for permanent insulin replacement (Lacy et al., 1986). Successful transplantation is difficult, however, because the pancreas is a fragile and complex organ. In addition, it is impossible for a human donor to give only a portion of a pancreas; thus the only feasible source is a deceased donor. Further, only a small portion of the pancreas, the β-cells of the islet of Langerhans, produce insulin. The remainder of the transplanted pancreas (i.e., non-β-cells) presents a potential target for transplant rejection. This is evident by the finding that transplanted islets are recognized and destroyed by the same autoimmune mechanisms responsible for destruction of the patients original islet β-cells. Thus, the transplant recipient must be subjected to a life-long regime of immunosuppressive drugs to prevent transplant rejection.

Genetically engineered insulinoma cell lines also have been developed as an alternative to isolated islets for transplantation therapy (U.S. Pat. Nos. 5,993,799; 5,792,656). These insulinoma cell lines can secrete insulin in response to glucose and thus potentially be introduced into a diabetic subject by transplantation. One obstacle to using such engineered insulinoma cell lines and other cell lines useful in transplantation for various other disease states will be the development of such cells that can withstand cytokine-mediated damage (e.g., IL-1β, IFN-γ) (Mandrup-Poulsen, 1996; Rabinovitch, 1993; Corbett and McDaniel, 1992; Eizirik et al., 1996). In one example, the cytotoxic effects of IL-1β against β-cell viability has been demonstrated, and methods such as the stable expression of MnSOD have been described which can reduce IL-1β induced β-cell cytotoxicity (Hohmeier et al., 1998).

The treatment of insulin-dependent diabetes by β-cell transplantation would be a significant improvement over current insulin injection treatments. However, current transplantation methods do not overcome the problems associated with transplant rejection and require further development of cell lines that can withstand cytokine-mediated damage of the cells. Accordingly, it is evident that improvements are still needed in the treatment of diabetes and cell transplantation in general.

### SUMMARY OF THE INVENTION

The present invention overcomes the deficiencies in the art by providing methods for preventing and overcoming cytokine-mediated cytotoxicity in a transplanted cell. The invention provides methods whereby one may treat a disease requiring cell-transplantation, such as diabetes, by transplanting a subject with insulinoma cells that express at least one STAT-1α polypeptide(s) which confers resistance to cellular cytokines involved in graft-rejection responses. Reference in this application to “cytokine-mediated cell killing” is intended as a reference to inflammatory cytokines and the list of particular cytokines provided below.

In one embodiment, the invention provides a method for preventing cytokine-mediated cytotoxicity of a cell introduced into a subject comprising: a) transferring into the cell an expression cassette comprising a polynucleotide encoding STAT-1α under the control of a promoter operable in eukaryotic cells; and b) administering the cell to the subject, wherein expression of STAT-1α prevents cytokine-mediated cytotoxicity of transplanted cells in the subject. The cytokine to which the cell is resistant is an inflammatory cytokine, for example, IL-1β and/or IFN-γ. Other cytokines for which protection is provided are TNF-α, TNF-β and TGF-β. In fact, the response to any inflammatory cytokine produced by activated peripheral blood mononuclear cells should be abrogated by use of STAT-1α.

Resistant is defined as any statistically significant improvement of the target cell's viability to cytokine-mediated cytotoxicity when compared to a comparable cell that does not express STAT-1α. Such resistance may be tested, for example, in any standard assay, e.g., an MTT viability assay. The degree of improvement may range from 2-, 3-, 4-, 5-, 10- or 100-times that of controls. It is envisioned that cell populations will be provided that are 50%, 75%, 80%, 85%, 90%, 95%, 96%, 97%, 98%, 99%, 99.5%, 99.9% and 100% resistant to cytokine-mediated cell killing.

In one aspect of the method, the promoter is not a native STAT-1α promoter. In another aspect, the promoter is an inducible promoter, a tissue specific promoter or a constitutive promoter. In other aspects, the expression cassette is contained in a replication competent vector. In specific aspects of the invention, the vector is a viral vector, e.g., an adenoviral vector, a retroviral vector, a vaccinia viral vector, an adeno-associated viral vector, a polyoma viral vector, a lentivirus vector or a herpesviral vector.

In one embodiment, the method of administering the cells to the subject comprises injection, including multiple injection. In another embodiment of the method administering the cells comprises encapsulating the cells in a semipermeable device, and implanting the device into the subject.

In another embodiment the invention describes a method for preventing cytokine-mediated cytotoxicity of a transplanted cell comprising administering to a transplant subject an expression cassette comprising a polynucleotide encoding STAT-1α under the control of a promoter operable in eukaryotic cells, wherein expression of STAT-1α prevents the cytotoxicity of the transplanted cell in the transplant subject. In one aspect of this method, the promoter is not a native STAT-1α promoter. In another aspect of this method, the promoter is an inducible promoter, a tissue specific promoter or a constitutive promoter.

The invention also contemplates a pharmaceutical composition comprising a polynucleotide encoding a STAT-1α and a non-STAT-1α promoter operable in eukaryotic cells, in a pharmaceutically acceptable carrier. In a related aspect of this embodiment, the STAT-1α comprises the sequence of SEQ ID NO:2. In another related aspect, the polynucleotide comprises the sequence of SEQ ID NO:1. The polynucleotide can further comprises a polyadenylation signal. In yet another related aspect, the polynucleotide is contained in a replication competent vector, e.g., viral vector or a plasmid vector. In one embodiment the promoter is an inducible promoter, a tissue specific promoter or a constitutive promoter.

The invention also provides, a cell comprising an expression construct comprising a polynucleotide encoding a STAT-1α polypeptide under the control of a non-STAT-1α promoter operable in eukaryotic cells, wherein the cell secretes insulin in response to glucose. In one embodiment, the STAT-1α comprises the sequence of SEQ ID NO:2. In another embodiment, the polynucleotide comprises the sequence of SEQ ID NO:1. In one aspect the polynucleotide further comprises a polyadenylation signal. In another aspect, the promoter is an inducible promoter, a tissue specific promoter or a constitutive promoter. In a related embodiment the cell is IL-1β resistant or IFN-γ resistant, or IL-1β and IFN-γ resistant.

The invention also describes a cell comprising an expression construct comprising a polynucleotide encoding a STAT-1α polypeptide under the control of a non-STAT-1α promoter operable in eukaryotic cells, wherein the cell produces and/or secretes GLP-1. In a related embodiment the invention discloses a cell comprising an expression construct comprising a polynucleotide encoding a STAT-1α polypeptide under the control of a non-STAT-1α promoter operable in eukaryotic cells, wherein the cell produces and/or secretes LCAT.

One embodiment of the invention is a polynucleotide encoding a human STAT-1α. In a specific embodiment, the STAT-1α has the amino acid sequence of SEQ ID NO:2. In another specific embodiment, the STAT-1α has the nucleotide sequence of SEQ ID NO:1. The invention also describes an expression construct comprising a polynucleotide encoding a rat STAT-1α operably linked to a promoter active in eukaryotic cells. The invention also further describes an expression construct comprising a polynucleotide encoding a mouse STAT-1α operably linked to a promoter active in eukaryotic cells. The expression constructs are further defined as a viral expression construct. The promoter of the expression construct is an inducible promoter, a constitutive promoter, a tissue specific promoter, or a native STAT-1α promoter.

Thus, the current invention describes the development of several methods for the generation of cells and cell lines that can withstand cytokine-mediated damage of the cells when transplanted into a host. These methods can be applied in any form of cell transplantation to prevent graft rejection by cytokine-mediated immunity, for example, the methods can be used for the treatment of diabetes by cell transplantation.

As used in the specification and claims the words “a” and “an” when used in combination with the conjunction “comprising” denote “one or more than one”.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B. Viability of parental and selected INS-1 cells following cytokine treatment. INS-1 cells were selected in increasing concentrations of IL-1β+IFN-γ (FIG. 1B) or were grown in normal tissue culture medium (FIG. 1A) for a period of 8 weeks. Following this procedure, cells were seeded in 96 well dishes and grown overnight in normal medium in the absence of cytokines. They were then cultured for 0-5 days in normal tissue culture medium containing no added cytokines (control), 10 mg/ml of rat IL-1β, 100 U/ml of rat IFN-γ, or both cytokines, as indicated in the figure legend. After this incubation, cells were subjected to the MTT viability assay, as described in Materials and Methods. Data represent the mean±SEM for 3 independent experiments, each performed in triplicate.

FIG. 2. Viability of parental and selected INS-1 cells following treatment with supernatants from activated PBMC. PBMC were prepared from normal Wistar rats and treated with 10 ng/ml PMA, 10 μg/ml LPS, or 10 μg/ml Con A. The conditioned media from these cells was then collected and added to parental INS-1 cells (white bars) or INS-1ᵣₑₛ cells (dark bars) for 48 h. Controls included application of medium alone (medium), medium supplemented with LPS (LPS), medium supplemented with PMA (PMA), medium supplemented with Con A (Con A), or medium from unstimulated rat PBMC (PBMC). The percentage of cells that were viable after these treatments was determined by the MTT assay as described in Example 1 and expressed as a percentage of viable cells following treatment with medium alone. Data represent the mean±SEM for 3 independent experiments, each performed in triplicate. The symbol * indicates conditions for which INS-1ᵣₑₛ cells are more viable than parental INS-1 cells, with p<0.001.

FIGS. 3A-B. Resistance to Cytokines and PBMC Supernatants is Partially Maintained in INS-1ᵣₑₛ Cells Cultured in the Absence of Cytokines for 6 Week. INS-1ᵣₑₛ cells were prepared by cytokine selection as described in Materials and Methods and then subjected to a 6 week period of growth in normal tissue culture medium lacking cytokines. At the end of this period, cells were treated with cytokines (FIG. 3A) or media from activated rat PBMC (FIG. 3B), as described for FIGS. 1 and 2. Data represent the mean±SEM for 3 independent experiments, each performed in triplicate.

FIG. 4. Levels of IL-1RI mRNA in parental INS-1 cells and INS-1ᵣₑₛ cells. Parental INS-1 cells, INS-1ᵣₑₛ cells grown in tissue culture medium containing IL-1β+IFN-γ, or INS-1ᵣₑₛ cells grown for 6 weeks in the absence of cytokines were subjected to Northern blot analysis, using the cDNA encoding IL-1RI (IL-1 receptor type I). The bottom panel is a loading control showing the 28S and 18S ribosomal bands. The middle panel shows the blot hybridized with the IL-1RI probe, and the top panel shows the phosphoimage quantification of the IL-1RI bands. Data in the top panel represent the mean±SEM of the representative experiment shown here (total number of experiments for each condition=3). The symbol * indicates that INS-1ᵣₑₛ cells cultured with or without cytokines contain significantly less IL-1RI mRNA, with p<0.001.

FIG. 5. Levels of IFN-γ receptor mRNA in parental INS-1 cells and INS-1ᵣₑₛ cells. Parental INS-1 cells, INS-1ᵣₑₛ cells grown in tissue culture medium containing IL-1β+IFN-γ, or INS-1ᵣₑₛ cells grown for 6 weeks in the absence of cytokines were subjected to Northern blot analysis, using the cDNA encoding the IFN-γ receptor. The bottom panel is a loading control showing the 28S and 18S ribosomal bands. The middle panel shows the blot hybridized with the IFN-γ receptor probe, and the top panel shows the phosphoimage quantification of the IFN-γ receptor bands. Data in the top panel represent the mean±SEM of the representative experiment shown (total number of experiments for each condition=3).

FIG. 6. Viability of clones derived by stable transfection of parental INS-1 cells or INS-1ᵣₑₛ cells. Parental INS-1 cells or INS-1ᵣₑₛ cells were stably transfected with a plasmid containing the cDNA encoding human insulin (12), resulting in the isolation of 58 and 51 individual colonies, respectively. These clones were grown individually in the absence of cytokines, and then treated for 48 h in the presence of 10 ng/ml or rat IL-1β+100 U/ml of rat IFN-γ. Viability was assessed by the MTT assay for all clones and is expressed as percent of viability of cells incubated for 48 h in the absence of cytokines. Each clone was assayed in triplicate, and data represent the mean±SEM of the 58 individual clones derived from parental INS-1 cells and the 51 individual clones derived the INS-1ᵣₑₛ. The symbol * indicates that clones derived from INS-1ᵣₑₛ were significantly more viable, with p<0.001.

FIG. 7. Effects of individual and combined cytokines on clones derived by stable transfection of parental INS-1 cells or INS-1ᵣₑₛ cells. Clonal lines derived by transfection as described in FIG. 6 and in the text were screened for glucose responsiveness. Nine clones derived from INS-1ᵣₑₛ cells and 8 clones from parental INS-1 cells with glucose responses of ≧5-fold were chosen for further study. The glucose responsive clones derived from parental INS-1 cells (white bars) or INS-1ᵣₑₛ cells (dark bars) were grown in the absence of cytokines, and then treated for 48 h in normal medium, or in normal medium containing 10 ng/ml of rat IL-1β, 100 U/ml or rat IFN-γ, or both cytokines. Viability was assessed by the MTT assay, and expressed as percent relative to control cells grown in normal medium. Each clone was assayed in triplicate, and data represent the mean±SEM of the 8 individual clones derived from parental INS-1 cells and the 9 individual clones derived from INS-1ᵣₑₛ. The symbol * indicates conditions for which clones derived from INS-1ᵣₑₛ were significantly more viable, with p<0.001.

FIG. 8. STAT-1 expression is increased in response to chronic treatment of INS-1 cells with IL-1β+IFN-γ. INS-1-derived (833/40) and INS-1ᵣₑₛ-derived (833/15, 833/117) clonal cell lines were grown in the presence or absence of IL-1β+IFN-γ (100 U/ml and 10 ng/ml, respectively) for a period of 48 h (culture condition) in 12 well plates. Cells were then shifted to serum free medium without cytokines overnight, treated with or without the indicated concentrations of cytokines for 20 min, washed with PBS and lysed. Fifty μg of total protein was loaded in each lane. Activated STAT-1 was detected by using anti-phospho-STAT-1 (Tyr701) antibody. Total STAT-1 was measured with an anti-STAT-1 antibody.

FIG. 9. IFN-γ-induced STAT-1 expression is inhibited by IL-1β in cytokine-sensitive INS-1 clone 834/40, but not in resistant clone 833-117. INS-1-derived (834/40) and INS-1ᵣₑₛ-derived (833/117) cells were cultured in normal medium lacking cytokines for a period of 6 months. Cells were then plated in 12 well plates and treated with IFN-γ (100 U/ml) or IFN-γ (100 U/ml)+IL-1β (10 ng/ml). At the indicated time, cells were washed with PBS and lysed in 200 μl of lysis buffer. Twenty-five μg of total protein was separated by 10% SDS-PAGE, electro-transferred, blotted with rabbit anti-STAT-1 antibody and visualized with enhanced chemiluminescence.

FIG. 10. Time course of decrease in STAT-1α expression after cytokine removal from culture medium of INS-1ᵣₑₛ clones. INS-1ᵣₑₛ clones (833/15 and 833/117) were inoculated into 12 well plates in the presence of IL-1β (10 ng/ml)+IFN-γ (100 U/ml) and cultured for 48 h. Cells were then washed with PBS (day 0) and cultured in medium with or without cytokines as indicated. At 2 and 6 days after the start of this culture period, cells were lysed in 200 μl of lysis buffer. Twenty-five μg of total protein per well was used for Western blot analysis of STAT-1 protein level.

FIGS. 11A-B. Adenovirus-mediated expression of STAT-1α in a cytokine-sensitive INS-1-derived cell line confers broad resistance to cytokine-induced cytotoxicity. FIG. 11A. INS-1 cells were treated with the indicated concentrations of AdCMV-STAT-1α for 12 h, and then incubated in fresh medium for 24 h prior to cell harvesting and immunoblot analysis with an anti-STAT-1α antibody. Control cells were incubated for the same time period but without virus treatment. FIG. 11B. The cytokine-sensitive cell line 834/40 was treated with 1×10⁹ pfu/ml of AdCMV-STAT1α, the same amount of AdCMV-βGAL virus, or no virus. These cell groups were cultured in normal medium for 24 hours, and then treated with 100 ng/ml IL-1β, 100 U/ml IFN-γ or both cytokines for 48 h. Cell viability was then determined with the MTT assay, as described in Materials and Methods. Data represent the mean±SEM for 4 independent experiments. Symbols refer to comparisons of viability of STAT-1α overexpressing cells to other groups, as follows: @, p<0.001, versus untreated control, p<0.03 versus AdCMV-βGAL-treated control; #, p=0.05 versus untreated control, p<0.007 versus AdCMV-βGAL-treated control; *, p<0.002 versus either control group.

FIG. 12. Adenovirus-mediated expression of STAT-1α in a cytokine-sensitive INS-1-derived cell line partially blocks cytokine-induced NO production. The cytokine-sensitive cell line 834/40 was treated exactly as described in FIG. 11, and media was collected for assay of nitrite levels. Data represent the mean±SEM for 5 independent experiments. The symbol * indicates that AdCMV-STAT-1α treated cells produced less nitrite than either control group, with p<0.01.

FIG. 13. Adenovirus-mediated expression of STAT-1α in a cytokine sensitive INS-1-derived cell line partially blocks cytokine-induced iNOS expression. The cytokine-sensitive cell line 834/40 was treated exactly as described in FIG. 11. Following 48 h of culture in the presence or absence of the indicated cytokines, cells were harvested for immunoblot analysis with an anti-iNOS antibody. Data from two independent experiments are shown.

FIG. 14. Overexpression of STAT-1α does not affect glucose-stimulated insulin secretion. The INS-1 derived cell line 83213 (16) was left untreated, or was treated with 1×10⁹ pful/ml of AdCMV-STAT-1α or AdCMV-βGAL adenoviruses. 36 h after viral treatment, cells were treated with 3 mM glucose or 15 mM glucose for 2 h, and media were collected for insulin radioimmunoassay. Data are normalized to total cell protein, and represent the mean±SEM for 3 independent experiments.

### DESCRIPTION OF ILLUSTRATIVE EMBODIMENTS

The present invention addresses the need for improved methods and compositions for preventing cytokine-mediated cytotoxicity of transplanted cells. More particularly, it provides methods and compositions useful in preventing interleukin-1β (IL-1β)- and interferon γ (IFN-γ)-mediated cytotoxicity of transplanted cells. Other inflammatory cytokines for which protection is provided are TNF-α, TNF-β, and TGF-β.

A large body of evidence has accumulated in support of the idea that inflammatory cytokines cause destruction of insulin-producing β cells of the islets of Langherhans in individuals with insulin-dependent diabetes mellitus (IDDM) (Mandrup-Poulsen, 1996; Rabinovitch, 1993; Corbett and McDaniel, 1992; Eizirik et al., 1996). Cytokines have also been reported to be cytotoxic to insulinoma cell lines, but almost all the work in this area has focused on the RINm5F cell line, which lacks glucose-stimulated insulin secretion and has a very low insulin content (Halban et al., 1983). The present inventors have developed insulinoma cell lines that produce insulin in response to glucose, that potentially can be used in transplant therapy for individuals with IDDM (U.S. Pat. Nos. 5,792,656; 5,993,799, each specifically incorporated herein by reference in its entirety). The inventors have additionally developed insulinoma cell lines that produce insulin in response to glucose and have improved resistance to IL-1β and IFN-γ induced cytotoxicity (described further in Examples 2-5).

Interleukin-1 (IL-1) is a cytokine produced by a variety of cell types including endothelial cells, fibroblasts, smooth muscle cells, keratinocytes, Langerhans cells of the skin, osteoclasts, astrocytes, epithelial cells of the thymus and the cornea, T-cells, B-cells and NK-cells, with the majority of the cytokine produced by cells of the monocyte/macrophage lineage. Interleukin 1 is secreted in two relatively functionally equivalent forms, IL-1α and IL-1β. As both IL-1α and IL-1β bind the same receptor with similar affinities, they possess similar if not identical biological activities. The two receptors for IL-1 (designated type 1 and type 2) are expressed predominantly on T-cells and cells of mesenchymal origin, and B-cells, granulocytes, and cells of the myelomonocytic lineage, respectively. Signal transducation by the IL1 receptor involves the adenylate cyclase mediated increase in intracellular cAMP levels. The binding of IL1 to its receptor further results in the activation of transcription factor NF-kappa-B and a short isoform of STAT-3 that potently stimulates transcription (Morton, et al. 1999). It is hypothesized that the activated STAT-3 and the IL-1 receptor accessory protein interact, which would suggest an association between the two IL1 receptor signaling pathways (Morton, et al. 1999).

IFN-γ is a cytokine that is released by antigen-stimulated T cells, which subsequently can induce the expression of numerous genes by binding to a specific receptor on the surface of most cells. IFN-γ is important in stimulating the function of various cells that participate in the immune response. Early experiments identified that IFN-γ induces the transcription of a gene encoding a guanylate binding protein by activating a latent, 91-kilodalton cytoplasmic protein, GAF (gamma-activated factor) (Shuai et al., 1992). It was subsequently observed that IFN-γ stimulated other genes by inducing tyrosine phosphorylation of the latent cytoplasmic factor GAF, now termed STAT (signal transducer and activator of transcription) (Shuai et al., 1993). STAT was observed to be phosphorylated on a single tyrosine residue (Tyr 701), and phosphorylation of this site was required for translocation of STAT from the cytoplasm to the nucleus, for DNA binding and gene activation.

It is demonstrated, in one embodiment of the present invention, that expressing a polynucleotide encoding a STAT-1α polypeptide in a cell, prevents IL-1β- and IFN-γ-cytotoxicity of the cell. Thus, in certain embodiments, the invention provides an expression cassette or a cell comprising a polynucleotide encoding a STAT-1α polypeptide for use in preventing IL-1β- and IFN-γ-mediated cytotoxicity of a cell introduced into a subject. In other embodiments, a cell line is provided that comprises a polynucleotide encoding a STAT-1α polypeptide and secretes insulin in response to glucose. It is contemplated that such a cell line will be useful in the treatment of insulin-dependent diabetes mellitus (IDDM) by cell transplantation.

More generally, the invention provides cells that are resistant to any cytokine that acts through a STAT-1α-mediated mechanism.

A. Nucleic Acids

The present invention provides STAT-1α polynucleotide compositions and methods for preventing IL-1β- and IFN-γ-mediated cytotoxicity of a cell introduced into a subject. The expression of a polynucleotide encoding a STAT-1α polypeptide in a cell, as demonstrated in the present invention, overcomes IL-1β- and IFN-γ-mediated cytotoxicity of the cell. It is therefore contemplated in specific embodiments of the invention that an expression cassette or a cell comprising a polynucleotide encoding a STAT-1α polypeptide will be used in preventing IL-1β- and IFN-γ-mediated cytotoxicity of a cell introduced into a subject by injection, transplantation and the like.

Human (SEQ ID NO:1) and mouse STAT-1α genes have been cloned and identified previously (Schindler et al., 1992; Zhong et al., 1994). The present inventors have cloned and identified a STAT-1α polynucleotide from rat (SEQ ID NO:3). The invention is not limited in scope to these genes, however, as one of ordinary skill in the art could, using these nucleic acids, readily identify related homologs in various other species. As discussed below, a specific “STAT-1α” gene or polynucleotide may contain a variety of different bases and yet still produce a corresponding polypeptide that is functionally indistinguishable, and in some cases structurally indistinguishable, from the polynucleotide sequences disclosed herein. Furthermore, a given STAT-1α polynucleotide from a given species may be represented by natural variants that have slightly different nucleic acid sequences but, nonetheless, encode the same polypeptide (see Table 2 below). Thus, it should be clear that the present invention is not limited to the specific nucleic acids disclosed herein.

1. Nucleic Acids Encoding STAT-1α Polypeptides

The present invention provides polynucleotides encoding STAT-1α polypeptides that prevent IL-1β- and IFN-γ-mediated cytotoxicity of a cell. The polynucleotide may be derived from genomic DNA or comprise complementary DNA (cDNA).

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression.

It also is contemplated that a given STAT-1α polynucleotide from a given species may be represented by natural variants that have slightly different nucleic acid sequences but, nonetheless, encode the same polypeptide (see Table 2 below). In addition, it is contemplated that a given STAT-1α polypeptide from a species may be generated using alternate codons that result in a different nucleic acid sequence but encodes the same polypeptide.

As used in this application, the term “a polynucleotide encoding a STAT-1α polypeptide” refers to a nucleic acid molecule that has been isolated free of total cellular nucleic acid. The term “functionally equivalent codon” is used herein to refer to codons that encode the same amino acid, such as the six codons for arginine or serine (Table 2, below), and also refers to codons that encode biologically equivalent amino acids, as discussed in the following pages.

TABLE 2

| | |
|---|---|
| Amino Acids | Codons |

| | | | | |
|---|---|---|---|---|
| Alanine | Ala | A | GCA GCC GCG | GCU |
| Cysteine | Cys | C | UGC UGU | |
| Aspartic acid | Asp | D | GAC GAU | |
| Glutamic acid | Glu | E | GAA GAG | |
| Phenylalanine | Phe | F | UUC UUU | |
| Glycine | Gly | G | GGA GGC GGG GGU | |
| Histidine | His | H | CAC CAU | |
| Isoleucine | Ile | I | AUA AUC AUU | |
| Lysine | Lys | K | AAA AAG | |
| Leucine | Leu | L | UUA UUG CUA CUC CUG CUU | |
| Methionine | Met | M | AUG | |
| Asparagine | Asn | N | AAC AAU | |
| Proline | Pro | P | CCA CCC CCG CCU | |
| Glutamine | Gln | Q | CAA CAG | |
| Arginine | Arg | R | AGA AGG CGA CGC CGG CGU | |
| Serine | Ser | S | AGC AGU UCA UCC UCG UCU | |
| Threonine | Thr | T | ACA ACC ACG ACU | |
| Valine | Val | V | GUA GUC GUG GUU | |
| Tryptophan | Trp | W | UGG | |
| Tyrosine | Tyr | Y | UAC UAU | |

Allowing for the degeneracy of the genetic code, sequences that have at least about 50%, usually at least about 60%, more usually about 70%, most usually about 80%, preferably at least about 90% and most preferably about 95% of nucleotides that are identical to the nucleotides of given STAT-1α gene or polynucleotide. Sequences that are essentially the same as those set forth in a STAT-1α gene or polynucleotide may also be functionally defined as sequences that are capable of hybridizing to a nucleic acid segment containing the complement of a STAT-1α polynucleotide under standard conditions.

The DNA segments of the present invention include those encoding biologically functional equivalent STAT-1α proteins and peptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed may be introduced through the application of site-directed mutagenesis techniques or may be introduced randomly and screened later for the desired function, as described below.

2. Oligonucleotide Probes and Primers

Naturally, the present invention also encompasses DNA segments that are complementary, or essentially complementary to the sequences of a STAT-1α polynucleotide. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of a STAT-1α polynucleotide under relatively stringent conditions such as those described herein. Such sequences may encode the entire STAT-1α polypeptide or functional or non-functional fragments thereof.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, 1000, 1212, 1500, 2000, 2500, 3000 or 3500 bases and longer are contemplated as well. Such oligonucleotides will find use, for example, as probes in Southern and Northern blots and as primers in amplification reactions.

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C. Formamide and SDS also may be used to alter the hybridization conditions.

One method of using probes and primers of the present invention is in the search for genes related to STAT-1α or, more particularly, homologs of STAT-1α from other species. Normally, the target DNA will be a genomic or cDNA library, although screening may involve analysis of RNA molecules. By varying the stringency of hybridization, and the region of the probe, different degrees of homology may be discovered.

Another way of exploiting probes and primers of the present invention is in site-directed, or site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

The technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

B. Cell Lines and Cell Culture

As used herein, the terms “cell,” “cell line,” and “cell culture” may be used interchangeably. All of these term also include their progeny, which is any and all subsequent generations. It is understood that all progeny may not be identical due to deliberate or inadvertent mutations. In the context of expressing a heterologous nucleic acid sequence such as a polynucleotide encoding a STAT-1α polypeptide or protein herein, “host cell” refers to eukaryotic cell, and it includes any transformable organisms that is capable of replicating a vector and or/expressing a heterologous gene encoded by a vector (Vectors are described in detail below). A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Examples of eukaryotic host cells for replication and/or expression of a vector include but are not limited to INS-1 cells, HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and/or their cognate polypeptides, proteins, or peptides.

The present invention provides methods and compositions for preventing IL-1β and IFN-γ-mediated cytotoxicity of cells. In particular embodiments, the invention provides ex vivo methods for preventing IL-1β- and IFN-γ-mediated cytotoxicity of a cell introduced into a subject. In these embodiments, an expression cassette comprising a polynucleotide encoding a STAT-1α polypeptide or protein under the control of a promoter operable in eukaryotic cells is introduced into a cell and the cell is administered to the subject.

In other embodiments cell compositions may have been engineered to secrete insulin, including those that respond to glucose, as described in U.S. Pat. Nos. 5,427,940, 5,854,067, 5,811,266, 5,993,799, and 5,792,656 (each specifically incorporated herein by reference). The above methods and compositions are contemplated to be particularly useful in the treatment of diabetes by cell transplantation. Also contemplated are cell compositions comprising a polynucleotide encoding a STAT-1α polypeptide or protein and secrete GLP-1, LCAT or various other proteins of interest. U.S. Ser. Nos. 08/589,028, 08/785,271, and 08/784,582 (each specifically incorporated herein by reference). Thus, the following is a general description of how to prepare such engineered cells for use in the present invention.

It has been demonstrated by the inventors that the inflammatory cytokines IL-1β and IFN-γ induce cytotoxicity in INS-1 cells. In the present invention, a selection strategy designed for obtaining INS-1 cells that are resistant to both of these cytokines is provided. It is contemplated, that when these cytokine-resistant cells, further comprise a polynucleotide encoding a STAT-1α polypeptide or protein according to the present invention, they will be particularly useful for preventing IL-1β- and IFN-γ-mediated cytotoxicity of cells. The general protocol for selection of INS-1 cells resistant to IL-1β and IFN-γ cytokines is described below.

INS-1 cells are cultured in a medium with increasing concentrations of IL-1β+IFN- γ over an 8 week period, beginning at 0.5 ng/ml +5 IU/ml, and ending at 10 ng/ml +100 IU/ml, respectively. The cells that grow well at the highest cytokine concentrations, are designated INS-1ᵣₑₛ. The degree of cytokine resistance achieved by this selection strategy is then investigated using an MTT cell viability assay. Parental INS-1 and INS- 1ᵣₑₛ cells are treated with I1-β (10 ng/ml), IFN-γ (100 U/ml) or IL-1β (10 ng/ml) +IFN-γ (100 IU/ml). The inventors found that in parental INS-1 cells, 1 day of treatment with IL- 1β or IFN-γ alone reduced viability to 40±3% and 68±3%, respectively, relative to untreated cells, with similar viabilities at longer time periods of treatment with these cytokines. The combination of IL-1β (10 ng/ml)+IFN-γ (100 U/ml was much more potent, causing a sharp drop to 21±0.4% viability after 1 day of exposure, and then a continued decline to near-complete cell destruction (0.4±0.03% viability) after 5 days. In sharp contrast, cell viabilities of INS-1ᵣₑₛ cells were maintained at 89±1.3%, 72±1.1%, and 71±2.6% at 2 days, and 100±1%, 82±1%, and 78±1% after 5 days of treatment with IL-1β, IFN-γ, or both cytokines, respectively. These data confirm that INS-1ᵣₑₛ cells have gained resistance to cytokine-induced cell damage.

Unfortunately, in the absence of continued selective pressure, the increase in STAT-1α expression is lost. Concommittant with this reduction in STAT-1α expression is the return in sensitivity to IFN-γ, but not IL-1β. Thus, the present invention seeks to establish a more permanent increase in STAT-1α expression to protect engineered cells, for example, those that secrete insulin in response glucose stimulation. Such cell lines are contemplated to be useful in the treatment of diabetes via cell transplantation, wherein the expression of STAT-1α prevents IL-1β- and IFN-γ-mediated cytotoxicity of the transplanted cells. In addition, the selection strategy for preparing the cytokine-resistant cells described above, could be implemented for an attenuated resistance to IL-1β- and IFN-γ-mediated cytotoxicity. The methods for preparing and using genetically engineered cells that produce insulin in response to glucose are described in detail in U.S. Pat. Nos. 5,792,656 and 5,993,799 (each specifically incorporated herein by reference).

C. Gene Delivery

In certain embodiments of the invention, an expression cassette comprising a STAT-1α gene or polynucleotide segment under the control of a heterologous promoter operable in eukaryotic cells is provided. The general approach in certain aspects of the present invention is to provide a cell with an expression construct encoding a STAT-1α that is capable of preventing IL-1β- and IFN-γ-mediated cytotoxicity of a cell. Following delivery of the expression construct, the protein encoded by the expression construct is synthesized by the transcriptional and translational machinery of the cell, as well as any that may be provided by the expression construct. Section B above describes cell lines contemplated to be particular useful in the present invention. The following section describes methods for the delivery of a STAT-1α polynucleotide into a cell for use in either ex vivo or in vivo applications of the invention.

In certain embodiments of the invention, the nucleic acid encoding the gene or polynucleotide is stably integrated into the genome of the cell. In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and/or where in the cell the nucleic acid remains is dependent on the type of vector employed.

Viral and non-viral vector systems are the two predominant categories for the delivery of an expression construct encoding a therapeutic protein or polypeptide. Both vector systems are described in the following sections. There also are two primary approaches utilized in the delivery of an expression construct for the purposes of gene therapy: either indirect, i.e., ex vivo methods; or direct, i.e., in vivo methods. Ex vivo gene transfer comprises vector modification of (host) cells in culture and the administration or transplantation of the vector modified cells to a gene therapy recipient. In vivo gene transfer comprises direct introduction of the vector (e.g., injection, inhalation) into the target source or therapeutic gene recipient.

The following gene delivery methods provide the framework for choosing and developing the most appropriate gene delivery system for a preferred application.

1. Non-Viral Polynucleotide Delivery

A STAT-1α expression construct may alternatively consist of naked recombinant DNA or plasmids. In certain embodiments of the invention, a STAT-1α expression construct is administered to a subject or a cell via injection. Injection of an expression construct may be administered by various routes of injection (e.g., intravenous, subcutaneous, intraperitoneal) as long as the expression construct can effectively be delivered to a desired target.

Transfer of a cloned expression construct in the present invention also may be performed by any of the methods which physically or chemically permeabilize the cell membrane (e.g., calcium phosphate precipitation, DEAE-dextran, electroporation, direct microinjection, DNA-loaded liposomes and lipofectamine-DNA complexes, cell sonication, gene bombardment using high velocity microprojectiles and receptor-mediated transfection. This is particularly applicable for transfer ex vivo or in vitro but it may be applied to in vivo use as well. Dubensky et al., (1984) successfully injected polyomavirus DNA in the form of calcium phosphate precipitates into liver and spleen of adult and newborn mice demonstrating active viral replication and acute infection. Benvenisty and Reshef (1986) also demonstrated that direct intraperitoneal injection of calcium phosphate-precipitated plasmids results in expression of the transfected genes. It is envisioned that DNA encoding a STAT-1α gene or polynucleotide of interest may also be transferred in a similar manner in vivo and express the gene or polynucleotide product.

a. Electroporation

In certain preferred embodiments of the present invention, the gene construct is introduced into the dendritic cells via electroporation. Electroporation involves the exposure of a suspension of cells and DNA to a high-voltage electric discharge.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

It is contemplated that electroporation conditions for dendritic cells from different sources may be optimized. One may particularly wish to optimize such parameters as the voltage, the capacitance, the time and the electroporation media composition. The execution of other routine adjustments will be known to those of skill in the art.

b. Particle Bombardment

Another embodiment of the invention for transferring a naked DNA construct into cells involves particle bombardment. This method depends on the ability to accelerate DNA-coated microprojectiles to a high velocity allowing them to pierce cell membranes and enter cells without killing them (Klein et al., 1987). The microprojectiles used have consisted of biologically inert substances such as tungsten, platinum or gold beads.

It is contemplated that in some instances DNA precipitation onto metal particles would not be necessary for DNA delivery to a recipient cell using particle bombardment. It is contemplated that particles may contain DNA rather than be coated with DNA. Hence it is proposed that DNA-coated particles may increase the level of DNA delivery via particle bombardment but are not, in and of themselves, necessary.

Several devices for accelerating small particles have been developed. One such device relies on a high voltage discharge to generate an electrical current, which in turn provides the motive force (Yang et al., 1990). Another method involves the use of a Biolistic Particle Delivery System, which can be used to propel particles coated with DNA through a screen, such as stainless steel or Nytex screen, onto a filter surface covered with cells in suspension. The screen disperses the particles so that they are not delivered to the recipient cells in large aggregates. It is believed that a screen intervening between the projectile apparatus and the cells to be bombarded reduces the size of projectile aggregates and may contribute to a higher frequency of transformation by reducing the damage inflicted on the recipient cells by projectiles that are too large.

For the bombardment, cells in suspension are preferably concentrated on filters, or alternatively on solid culture medium. The cells to be bombarded are positioned at an appropriate distance below the macroprojectile stopping plate. If desired, one or more screens are also positioned between the acceleration device and the cells to be bombarded.

In bombardment transformation, one may optimize the prebombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity or either the macro- or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmids. It is believed that pre-bombardment manipulations are especially important for successful transformation of primordial germ cells.

Accordingly, it is contemplated that one may wish to adjust various of the bombardment parameters in small scale studies to fully optimize the conditions. One may particularly wish to adjust physical parameters such as gap distance, flight distance, tissue distance and helium pressure. One may also optimize the trauma reduction factors by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. The execution of other routine adjustments will be known to those of skill in the art.

C. Calcium Phosphate Co-Precipitation or DEAE-Dextran Treatment

In other embodiments of the present invention, the transgenic construct is introduced to the cells using calcium phosphate co-precipitation. Mouse primordial germ cells have been transfected with the SV40 large T antigen, with excellent results (Watanabe et al., 1997). Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

In another embodiment, the expression construct is delivered into the cell using DEAE-dextran followed by polyethylene glycol. In this manner, reporter plasmids were introduced into mouse myeloma and erythroleukemia cells (Gopal, 1985).

d. Direct Microinjection or Sonication Loading

Further embodiments of the present invention include the introduction of the gene construct by direct microinjection or sonication loading. Direct microinjection has been used to introduce nucleic acid constructs into Xenopus oocytes (Harland and Weintraub, 1985), and LTK fibroblasts have been transfected with the thymidine kinase gene by sonication loading (Fechheimer et al., 1987).

e. Liposome-Mediated Transformation

In a further embodiment of the invention, the gene construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL).

Liposome-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of liposome-mediated delivery and expression of foreign DNA in cultured chick embryo, HeLa and hepatoma cells.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1.

2. Viral Vectors

In other embodiments, it is contemplated that a STAT-1α expression construct, that prevents IL-1β- and IFN-γ-mediated cytotoxicity, may be delivered by a viral vector. The capacity of certain viral vectors to efficiently infect or enter cells, to integrate into a host cell genome and stably express viral genes, have led to the development and application of a number of different viral vector systems (Robbins et al., 1998). Viral systems are currently being developed for use as vectors for ex vivo and in vivo gene transfer. For example, adenovirus, herpes-simplex virus, retrovirus and adeno-associated virus vectors are being evaluated currently for treatment of diseases such as cancer, cystic fibrosis, Gaucher disease, renal disease and arthritis (Robbins and Ghivizzani, 1998; Imai et al., 1998; U.S. Pat. No. 5,670,488). The various viral vectors described below, present specific advantages and disadvantages, depending on the particular gene-therapeutic application.

a. Adenoviral Vectors

In particular embodiments, an adenoviral expression vector is contemplated for the delivery of expression constructs. “Adenovirus expression vector” is meant to include those constructs containing adenovirus sequences sufficient to (a) support packaging of the construct and (b) to ultimately express a tissue or cell-specific construct that has been cloned therein.

Adenoviruses comprise linear, double-stranded DNA, with a genome ranging from 30 to 35 kb in size (Reddy et al., 1998; Morrison et al., 1997; Chillon et al., 1999). An adenovirus expression vector according to the present invention comprises a genetically engineered form of the adenovirus. Advantages of adenoviral gene transfer include the ability to infect a wide variety of cell types, including non-dividing cells, a mid-sized genome, ease of manipulation, high infectivity and the ability to be grown to high titers (Wilson, 1996). Further, adenoviral infection of host cells does not result in chromosomal integration because adenoviral DNA can replicate in an episomal manner, without potential genotoxicity associated with other viral vectors. Adenoviruses also are structurally stable (Marienfeld et al., 1999) and no genome rearrangement has been detected after extensive amplification (Parks et al., 1997; Bett et al., 1993).

Salient features of the adenovirus genome are an early region (E1, E2, E3 and E4 genes), an intermediate region (pIX gene, Iva2 gene), a late region (L1, L2, L3, L4 and L5 genes), a major late promoter (MLP), inverted-terminal-repeats (ITRs) and a ψ sequence (Zheng, et al., 1999; Robbins et al., 1998; Graham and Prevec, 1995). The early genes E1, E2, E3 and E4 are expressed from the virus after infection and encode polypeptides that regulate viral gene expression, cellular gene expression, viral replication, and inhibition of cellular apoptosis. Further on during viral infection, the MLP is activated, resulting in the expression of the late (L) genes, encoding polypeptides required for adenovirus encapsidation. The intermediate region encodes components of the adenoviral capsid. Adenoviral inverted terminal repeats (ITRs; 100-200 bp in length), are cis elements, and function as origins of replication and are necessary for viral DNA replication. The v sequence is required for the packaging of the adenoviral genome.

A common approach for generating an adenoviruses for use as a gene transfer vector is the deletion of the El gene (E1⁻), which is involved in the induction of the E2, E3 and E4 promoters (Graham and Prevec, 1995). Subsequently, a therapeutic gene or genes can be inserted recombinantly in place of the E1 gene, wherein expression of the therapeutic gene(s) is driven by the El promoter or a heterologous promoter. The E1⁻, replication-deficient virus is then proliferated in a “helper” cell line that provides the E1 polypeptides in trans (e.g., the human embryonic kidney cell line 293). Thus, in the present invention it may be convenient to introduce the transforming construct at the position from which the E1-coding sequences have been removed. However, the position of insertion of the construct within the adenovirus sequences is not critical to the invention. Alternatively, the E3 region, portions of the E4 region or both may be deleted, wherein a heterologous nucleic acid sequence under the control of a promoter operable in eukaryotic cells is inserted into the adenovirus genome for use in gene transfer (U.S. Pat. No. 5,670,488; 5,932,210, each specifically incorporated herein by reference).

Although adenovirus based vectors offer several unique advantages over other vector systems, they often are limited by vector immunogenicity, size constraints for insertion of recombinant genes and low levels of replication. The preparation of a recombinant adenovirus vector deleted of all open reading frames, comprising a full length dystrophin gene and the terminal repeats required for replication (Haecker et al., 1997) offers some potentially promising advantages to the above mentioned adenoviral shortcomings. The vector was grown to high titer with a helper virus in 293 cells and was capable of efficiently transducing dystrophin in mdx mice, in myotubes in vitro and muscle fibers in vivo. Helper-dependent viral vectors are discussed below.

A major concern in using adenoviral vectors is the generation of a replication-competent virus during vector production in a packaging cell line or during gene therapy treatment of an individual. The generation of a replication-competent virus could pose serious threat of an unintended viral infection and pathological consequences for the patient. Armentano et al., describe the preparation of a replication-defective adenovirus vector, claimed to eliminate the potential for the inadvertent generation of a replication-competent adenovirus (U.S. Pat. No. 5,824,544, specifically incorporated herein by reference). The replication-defective adenovirus method comprises a deleted E1 region and a relocated protein IX gene, wherein the vector expresses a heterologous, mammalian gene.

Other than the requirement that the adenovirus vector be replication defective, or at least conditionally defective, the nature of the adenovirus vector is not believed to be D-crucial to the successful practice of the invention. The adenovirus may be of any of the 42 different known serotypes and/or subgroups A-F. Adenovirus type 5 of subgroup C is the preferred starting material in order to obtain the conditional replication-defective adenovirus vector for use in the present invention. This is because adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Adenovirus growth and manipulation is known to those of skill in the art, and exhibits broad host range in vitro and in vivo (U.S. Pat. Nos. 5,670,488; 5,932,210; 5,824,54). This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. Many experiments, innovations, preclinical studies and clinical trials are currently under investigation for the use of adenoviruses as gene delivery vectors. For example, adenoviral gene delivery-based based gene therapies are being developed for liver diseases (Han et al., 1999), psychiatric diseases (Lesch, 1999), neurological diseases (Smith, 1998; Hermens and Verhaagen, 1998), coronary diseases (Feldman et al., 1996), muscular diseases (Petrof, 1998), gastrointestinal diseases (Wu, 1998) and various cancers such as colorectal (Fujiwara and Tanaka, 1998; Dorai et al., 1999), pancreatic (Carrion et al., 1999), bladder (Irie et al., 1999), head and neck (Blackwell et al., 1999), breast (Stewart et al., 1999), lung (Batra et al., 1999) and ovarian (Vanderkwaak et al., 1999).

b. Retroviral Vectors

In certain embodiments of the invention, the use of retroviruses for gene delivery are contemplated. Retroviruses are RNA viruses comprising an RNA genome. When a host cell is infected by a retrovirus, the genomic RNA is reverse transcribed into a DNA intermediate which is integrated into the chromosomal DNA of infected cells. This integrated DNA intermediate is referred to as a provirus. A particular advantage of retroviruses is that they can stably infect dividing cells with a gene of interest (e.g., a therapeutic gene) by integrating into the host DNA, without expressing immunogenic viral proteins. Theoretically, the integrated retroviral vector will be maintained for the life of the infected host cell, expressing the gene of interest.

The retroviral genome and the proviral DNA have three genes: gag, pol, and env, which are flanked by two long terminal repeat (LTR) sequences. The gag gene encodes the internal structural (matrix, capsid, and nucleocapsid) proteins; the pol gene encodes the RNA-directed DNA polymerase (reverse transcriptase) and the env gene encodes viral envelope glycoproteins. The 5′ and 3′ LTRs serve to promote transcription and polyadenylation of the virion RNAs. The LTR contains all other cis-acting sequences necessary for viral replication.

A recombinant retrovirus of the present invention may be genetically modified in such a way that some of the structural, infectious genes of the native virus have been removed and replaced instead with a nucleic acid sequence to be delivered to a target cell (U.S. Pat. Nos. 5,858,744; 5,739,018, each incorporated herein by reference). After infection of a cell by the virus, the virus injects its nucleic acid into the cell and the retrovirus genetic material can integrate into the host cell genome. The transferred retrovirus genetic material is then transcribed and translated into proteins within the host cell. As with other viral vector systems, the generation of a replication-competent retrovirus during vector production or during therapy is a major concern. Retroviral vectors suitable for use in the present invention are generally defective retroviral vectors that are capable of infecting the target cell, reverse transcribing their RNA genomes, and integrating the reverse transcribed DNA into the target cell genome, but are incapable of replicating within the target cell to produce infectious retroviral particles (e.g., the retroviral genome transferred into the target cell is defective in gag, the gene encoding virion structural proteins, and/or in pol, the gene encoding reverse transcriptase). Thus, transcription of the provirus and assembly into infectious virus occurs in the presence of an appropriate helper virus or in a cell line containing appropriate sequences enabling encapsidation without coincident production of a contaminating helper virus.

The growth and maintenance of retroviruses is known in the art (U.S. Pat. Nos. 5,955,331; 5,888,502, each specifically incorporated herein by reference). Nolan et al. describe the production of stable high titre, helper-free retrovirus comprising a heterologous gene (U.S. Pat. No. 5,830,725, specifically incorporated herein by reference). Methods for constructing packaging cell lines useful for the generation of helper-free recombinant retroviruses with amphoteric or ecotrophic host ranges, as well as methods of using the recombinant retroviruses to introduce a gene of interest into eukaryotic cells in vivo and in vitro are contemplated in the present invention (U.S. Pat. No. 5,955,331).

Currently, the majority of all clinical trials for vector-mediated gene delivery use murine leukemia virus (MLV)-based retroviral vector gene delivery (Robbins et al., 1998; Miller et al., 1993). Disadvantages of retroviral gene delivery includes a requirement for ongoing cell division for stable infection and a coding capacity that prevents the delivery of large genes. However, recent development of vectors such as lentivirus (e.g., HIV), simian immunodeficiency virus (SIV) and equine infectious-anemia virus (EIAV), which can infect certain non-dividing cells, potentially allow the in vivo use of retroviral vectors for gene therapy applications (Amado and Chen, 1999; Klimatcheva et al., 1999; White et al., 1999; Case et al., 1999). For example, HIV-based vectors have been used to infect non-dividing cells such as neurons (Takashi et al., 1999; Miyake et al., 1999), islets (Leibowitz et al., 1999) and muscle cells (Johnston et al., 1999). The therapeutic delivery of genes via retroviruses are currently being assessed for the treatment of various disorders such as inflammatory disease (Moldawer et al., 1999), AIDS (Amado et al., 1999; Engel and Kohn, 1999), cancer (Clay et al., 1999), cerebrovascular disease (Weihl et al., 1999) and hemophilia (Kay, 1998).

C. Herpes-Simplex Viral Vectors Herpes simplex virus (HSV) type I and type II contain a double-stranded, linear DNA genome of approximately 150 kb, encoding 70-80 genes. Wild type HSV are able to infect cells lytically and to establish latency in certain cell types (e.g., neurons). Similar to adenovirus, HSV also can infect a variety of cell types including muscle (Yeung et al., 1999), ear (Derby et al., 1999), eye (Kaufmnan et al., 1999), tumors (Yoon et al., 1999; Howard et al., 1999), lung (Kohut et al., 1998), neuronal (Garrido et al, 1999; Lachmann and Efstathiou, 1999), liver (Miytake et al., 1999; Kooby et al., 1999) and pancreatic islets (Rabinovitch et al., 1999).

HSV viral genes are transcribed by cellular RNA polymerase II and are temporally regulated, resulting in the transcription and subsequent synthesis of gene products in roughly three discernable phases or kinetic classes. These phases of genes are referred to as the Immediate Early (IE) or alpha genes, Early (E) or beta genes and Late (L) or gamma genes. Immediately following the arrival of the genome of a virus in the nucleus of a newly infected cell, the IE genes are transcribed. The efficient expression of these genes does not require prior viral protein synthesis. The products of IE genes are required to activate transcription and regulate the remainder of the viral genome.

For use in therapeutic gene delivery, HSV must be rendered replication-defective. Protocols for generating replication-defective HSV helper virus-free cell lines have been described (U.S. Pat. Nos. 5,879,934; 5,851,826, each specifically incorporated herein by reference in its entirety). One IE protein, Infected Cell Polypeptide 4 (ICP4), also known as alpha 4 or Vmw175, is absolutely required for both virus infectivity and the transition from IE to later transcription. Thus, due to its complex, multifunctional nature and central role in the regulation of HSV gene expression, ICP4 has typically been the target of HSV genetic studies.

Phenotypic studies of HSV viruses deleted of ICP4 indicate that such viruses will be potentially useful for gene transfer purposes (Krisky et al., 1998a). One property of viruses deleted for ICP4 that makes them desirable for gene transfer is that they only express the five other IE genes: ICP0, ICP6, ICP27, ICP22 and ICP47 (DeLuca et al., 1985), without the expression of viral genes encoding proteins that direct viral DNA synthesis, as well as the structural proteins of the virus. This property is desirable for minimizing possible deleterious effects on host cell metabolism or an immune response following gene transfer. Further deletion of IE genes ICP22 and ICP27, in addition to ICP4, substantially improve reduction of HSV cytotoxicity and prevented early and late viral gene expression (Krisky et al., 1998b).

The therapeutic potential of HSV in gene transfer has been demonstrated in various in vitro model systems and in vivo for diseases such as Parkinson's (Yamada et al., 1999), retinoblastoma (Hayashi et al., 1999), intracerebral and intradermal tumors (Moriuchi et al., 1998), B cell malignancies (Suzuki et al., 1998), ovarian cancer (Wang et al., 1998) and Duchenne muscular dystrophy (Huard et al., 1997).

d. Adeno-associated Viral Vectors

Adeno-associated virus (AAV), a member of the parvovirus family, is a human virus that is increasingly being used for gene delivery therapeutics. AAV has several advantageous features not found in other viral systems. First, AAV can infect a wide range of host cells, including non-dividing cells. Second, AAV can infect cells from different species. Third, AAV has not been associated with any human or animal disease and does not appear to alter the biological properties of the host cell upon integration. For example, it is estimated that 80-85% of the human population has been exposed to AAV. Finally, AAV is stable at a wide range of physical and chemical conditions which lends itself to production, storage and transportation requirements.

The AAV genome is a linear, single-stranded DNA molecule containing 4681 nucleotides. The AAV genome generally comprises an internal non-repeating genome flanked on each end by inverted terminal repeats (ITRs) of approximately 145 bp in length. The ITRs have multiple functions, including origins of DNA replication, and as packaging signals for the viral genome. The internal non-repeated portion of the genome includes two large open reading frames, known as the AAV replication (rep) and capsid (cap) genes. The rep and cap genes code for viral proteins that allow the virus to replicate and package the viral genome into a virion. A family of at least four viral proteins are expressed from the AAV rep region, Rep 78, Rep 68, Rep 52, and Rep 40, named according to their apparent molecular weight. The AAV cap region encodes at least three proteins, VP1, VP2, and VP3.

AAV is a helper-dependent virus requiring co-infection with a helper virus (e.g., adenovirus, herpesvirus or vaccinia) in order to form AAV virions. In the absence of co-infection with a helper virus, AAV establishes a latent state in which the viral genome inserts into a host cell chromosome, but infectious virions are not produced. Subsequent infection by a helper virus “rescues” the integrated genome, allowing it to replicate and package its genome into infectious AAV virions. Although AAV can infect cells from different species, the helper virus must be of the same species as the host cell (e.g., human AAV will replicate in canine cells co-infected with a canine adenovirus).

AAV has been engineered to deliver genes of interest by deleting the internal non-repeating portion of the AAV genome and inserting a heterologous gene between the ITRs. The heterologous gene may be functionally linked to a heterologous promoter (constitutive, cell-specific, or inducible) capable of driving gene expression in target cells. To produce infectious recombinant AAV (rAAV) containing a heterologous gene, a suitable producer cell line is transfected with a rAAV vector containing a heterologous gene. The producer cell is concurrently transfected with a second plasmid harboring the AAV rep and cap genes under the control of their respective endogenous promoters or heterologous promoters. Finally, the producer cell is infected with a helper virus.

Once these factors come together, the heterologous gene is replicated and packaged as though it were a wild-type AAV genome. When target cells are infected with the resulting rAAV virions, the heterologous gene enters and is expressed in the target cells. Because the target cells lack the rep and cap genes and the adenovirus helper genes, the rAAV cannot further replicate, package or form wild-type AAV.

The use of helper virus, however, presents a number of problems. First, the use of adenovirus in a rAAV production system causes the host cells to produce both rAAV and infectious adenovirus. The contaminating infectious adenovirus can be inactivated by heat treatment (56° C. for 1 hour). Heat treatment, however, results in approximately a 50% drop in the titer of functional rAAV virions. Second, varying amounts of adenovirus proteins are present in these preparations. For example, approximately 50% or greater of the total protein obtained in such rAAV virion preparations is free adenovirus fiber protein. If not completely removed, these adenovirus proteins have the potential of eliciting an immune response from the patient. Third, AAV vector production methods which employ a helper virus require the use and manipulation of large amounts of high titer infectious helper virus, which presents a number of health and safety concerns, particularly in regard to the use of a herpesvirus. Fourth, concomitant production of helper virus particles in rAAV virion producing cells diverts large amounts of host cellular resources away from rAAV virion production, potentially resulting in lower rAAV virion yields.

e. Lentiviral Vectors

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif vpr, vpu and nef are deleted making the vector biologically safe.

Recombinant lentiviral vectors are capable of infecting non-dividing cells and can be used for both in vivo and ex vivo gene transfer and expression of nucleic acid sequences. The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTR's serve to promote transcription and polyadenylation of the virion RNA's. The LTR contains all other cis-acting sequences necessary for viral replication. Lentiviruses have additional genes including vif, vpr, tat, rev, vpu, nef and vpx.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

Lentiviral vectors are known in the art, see Naldini et al., (1996); Zufferey et al, (1997); U.S. Pat. Nos. 6,013,516; and 5,994,136. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest.

Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene, such as the STAT-1α gene in this invention, into that packaging cell yields a producer cell which releases infectious viral particles carrying the foreign gene of interest. The env preferably is an amphotropic envelope protein which allows transduction of cells of human and other species.

One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

The vector providing the viral env nucleic acid sequence is associated operably with regulatory sequences, e.g., a promoter or enhancer. The regulatory sequence can be any eukaryotic promoter or enhancer, including for example, the Moloney murine leukemia virus promoter-enhancer element, the human cytomegalovirus enhancer or the vaccinia P7.5 promoter. In some cases, such as the Moloney murine leukemia virus promoter-enhancer element, the promoter-enhancer elements are located within or adjacent to the LTR sequences.

The heterologous or foreign nucleic acid sequence, such as the STAT-1α encoding polynucleotide sequence herein, is linked operably to a regulatory nucleic acid sequence. Preferably, the heterologous sequence is linked to a promoter, resulting in a chimeric gene. The heterologous nucleic acid sequence may also be under control of either the viral LTR promoter-enhancer signals or of an internal promoter, and retained signals within the retroviral LTR can still bring about efficient expression of the transgene. Marker genes may be utilized to assay for the presence of the vector, and thus, to confirm infection and integration. The presence of a marker gene ensures the selection and growth of only those host cells which express the inserts. Typical selection genes encode proteins that confer resistance to antibiotics and other toxic substances, e.g., histidinol, puromycin, hygromycin, neomycin, methotrexate etc. and cell surface markers.

The vectors are introduced via transfection or infection into the packaging cell line. The packaging cell line produces viral particles that contain the vector genome. Methods for transfection or infection are well known by those of skill in the art. After cotransfection of the packaging vectors and the transfer vector to the packaging cell line, the recombinant virus is recovered from the culture media and titered by standard methods used by those of skill in the art. Thus, the packaging constructs can be introduced into human cell lines by calcium phosphate transfection, lipofection or electroporation, generally together with a dominant selectable marker, such as neo, DHFR, Gln synthetase or ADA, followed by selection in the presence of the appropriate drug and isolation of clones. The selectable marker gene can be linked physically to the packaging genes in the construct.

Lentiviral transfer vectors Naldini et al., (1996), have been used to infect human cells growth-arrested in vitro and to transduce neurons after direct injection into the brain of adult rats. The vector was efficient at transferring marker genes in vivo into the neurons and long term expression in the absence of detectable pathology was achieved. Animals analyzed ten months after a single injection of the vector showed no decrease in the average level of transgene expression and no sign of tissue pathology or immune reaction (Blomer et al., (1997). Thus, in the present invention, one may graft or transplant cells infected with the recombinant lentivirus ex vivo, or infect cells in vivo.

f. Other Viral Vectors

The development and utility of viral vectors for gene delivery is constantly improving and evolving. Other viral vectors such as poxvirus; e.g., vaccinia virus (Gnant et al., 1999; Gnant et al., 1999), alpha virus; e.g., sindbis virus, Semliki forest virus (Lundstrom, 1999), reovirus (Coffey et al., 1998) and influenza A virus (Neumann et al., 1999) are contemplated for use in the present invention and may be selected according to the requisite properties of the target system.

In certain embodiments, vaccinia viral vectors are contemplated for use in the present invention. Vaccinia virus is a particularly useful eukaryotic viral vector system for expressing heterologous genes. For example, when recombinant vaccinia virus is properly engineered, the proteins are synthesized, processed and transported to the plasma membrane. Vaccinia viruses as gene delivery vectors have recently been demonstrated to transfer genes to human tumor cells, e.g., EMAP-II (Gnant et al., 1999), inner ear (Derby et al., 1999), glioma cells, e.g., p53 (Timiryasova et al., 1999) and various mammalian cells, e.g., P-450 (U.S. Pat. No. 5,506,138). The preparation, growth and manipulation of vaccinia viruses are described in U.S. Pat. Nos. 5,849,304 and 5,506,138 (each specifically incorporated herein by reference).

In other embodiments, sindbis viral vectors are contemplated for use in gene delivery. Sindbis virus is a species of the alphavirus genus (Garoff and Li, 1998) which includes such important pathogens as Venezuelan, Western and Eastern equine encephalitis viruses (Sawai et al., 1999; Mastrangelo et al., 1999). In vitro, sindbis virus infects a variety of avian, mammalian, reptilian, and amphibian cells. The genome of sindbis virus consists of a single molecule of single-stranded RNA, 11,703 nucleotides in length. The genomic RNA is infectious, is capped at the 5′ terminus and polyadenylated at the 3′ terminus, and serves as mRNA. Translation of a vaccinia virus 26S mRNA produces a polyprotein that is cleaved co- and post-translationally by a combination of viral and presumably host-encoded proteases to give the three virus structural proteins, a capsid protein (C) and the two envelope glycoproteins (E1 and PE2, precursors of the virion E2).

Three features of sindbis virus suggest that it would be a useful vector for the expression of heterologous genes. First, its wide host range, both in nature and in the laboratory. Second, gene expression occurs in the cytoplasm of the host cell and is rapid and efficient. Third, temperature-sensitive mutations in RNA synthesis are available that may be used to modulate the expression of heterologous coding sequences by simply shifting cultures to the non-permissive temperature at various time after infection. The growth and maintenance of sindbis virus is known in the art (U.S. Pat. No. 5,217,879, specifically incorporated herein by reference).

g. Chimeric Viral Vectors

Chimeric or hybrid viral vectors are being developed for use in therapeutic gene delivery and are contemplated for use in the present invention. Chimeric poxviral/retroviral vectors (Holzer et al., 1999), adenoviral/retroviral vectors (Feng et al., 1997; Bilbao et al., 1997; Caplen et al., 1999) and adenoviral/adeno-associated viral vectors (Fisher et al., 1996; U.S. Pat. No. 5,871,982) have been described.

These “chimeric” viral gene transfer systems can exploit the favorable features of two or more parent viral species. For example, Wilson et al., provide a chimeric vector construct which comprises a portion of an adenovirus, AAV 5′ and 3′ ITR sequences and a selected transgene, described below (U.S. Pat. No. 5,871,983, specifically incorporate herein by reference).

The adenovirus/AAV chimeric virus uses adenovirus nucleic acid sequences as a shuttle to deliver a recombinant AAV/transgene genome to a target cell. The adenovirus nucleic acid sequences employed in the hybrid vector can range from a minimum sequence amount, which requires the use of a helper virus to produce the hybrid virus particle, to only selected deletions of adenovirus genes, which deleted gene products can be supplied in the hybrid viral production process by a selected packaging cell. At a minimum, the adenovirus nucleic acid sequences employed in the pAdA shuttle vector are adenovirus genomic sequences from which all viral genes are deleted and which contain only those adenovirus sequences required for packaging adenoviral genomic DNA into a preformed capsid head. More specifically, the adenovirus sequences employed are the cis-acting 5′ and 3′ inverted terminal repeat (ITR) sequences of an adenovirus (which function as origins of replication) and the native 5′ packaging/enhancer domain, that contains sequences necessary for packaging linear Ad genomes and enhancer elements for the E1 promoter. The adenovirus sequences may be modified to contain desired deletions, substitutions, or mutations, provided that the desired function is not eliminated.

The AAV sequences useful in the above chimeric vector are the viral sequences from which the rep and cap polypeptide encoding sequences are deleted. More specifically, the AAV sequences employed are the cis-acting 5′ and 3′ inverted terminal repeat (ITR) sequences. These chimeras are characterized by high titer transgene delivery to a host cell and the ability to stably integrate the transgene into the host cell chromosome (U.S. Pat. No. 5,871,983, specifically incorporate herein by reference). In the hybrid vector construct, the AAV sequences are flanked by the selected adenovirus sequences discussed above. The 5′ and 3′ AAV ITR sequences themselves flank a selected transgene sequence and associated regulatory elements, described below. Thus, the sequence formed by the transgene and flanking 5′ and 3′ AAV sequences may be inserted at any deletion site in the adenovirus sequences of the vector. For example, the AAV sequences are desirably inserted at the site of the deleted E1a/E1b genes of the adenovirus. Alternatively, the AAV sequences may be inserted at an E3 deletion, E2a deletion, and so on. If only the adenovirus 5′ ITR/packaging sequences and 3′ ITR sequences are used in the hybrid virus, the AAV sequences are inserted between them.

The transgene sequence of the vector and recombinant virus can be a gene, a nucleic acid sequence or reverse transcript thereof, heterologous to the adenovirus sequence, which encodes a protein, polypeptide or peptide fragment of interest. The transgene is operatively linked to regulatory components in a manner which permits transgene transcription. The composition of the transgene sequence will depend upon the use to which the resulting hybrid vector will be put. For example, one type of transgene sequence includes a therapeutic gene which expresses a desired gene product in a host cell. These therapeutic genes or nucleic acid sequences typically encode products for administration and expression in a patient in vivo or ex vivo to replace or correct an inherited or non-inherited genetic defect or treat an epigenetic disorder or disease.

3. Vectors and Regulatory Signals

Vectors of the present invention are designed, primarily, to transform cells with a STAT-1α polynucleotide under the control of regulated eukaryotic promoters (i.e., inducible, repressable, tissue specific). Also, the vectors usually will contain a selectable marker if, for no other reason, to facilitate their production in vitro. However, selectable markers may play an important role in producing recombinant cells and thus a discussion of promoters is useful here. Table 2 and Table 3 below, list inducible promoter elements and enhancer elements, respectively.

a. Vector Promoters and Enhancers

Various promoter and enhancer elements are contemplated as useful in the present invention. Below are a list of viral promoters, cellular promoters/enhancers and inducible promoters/enhancers that could be used in combination with the present invention. Additionally any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest.

TABLE 2
Inducible Elements

| Element | Inducer | References |
|---|---|---|
| MT II | Phorbol Ester (TFA) | Palmiter et al., 1982; |
| | Heavy metals | Haslinger and Karin, |
| | | 1985; Searle et al., 1985; |
| | | Stuart et al., 1985; |
| | | Imagawa et al., 1987; |
| | | Karin ®, 1987; Angel et |
| | | al., 1987b; McNeall et |
| | | al., 1989 |
| MMTV (mouse | Glucocorticoids | Huang et al., 1981; Lee |
| mammary tumor virus) | | et al., 1981; Majors and |
| | | Varmus, 1983; Chandler |
| | | et al., 1983; Lee et al., |
| | | 1984; Fonta et al., 1985; |
| | | Sakai et al., 1986 |
| β-Interferon | poly(rI)X | Tavernier et al., 1983 |
| | poly(rc) | |
| Adenovirus 5 E2 | Ela | Imperiale and Nevins, |
| | | 1984 |
| Collagenase | Phorbol Ester (TPA) | Angle et al., 1987a |
| Stromelysin | Phorbol Ester (TPA) | Angle et al., 1987b |
| SV40 | Phorbol Ester (TFA) | Angel et al., 1987b |
| Murine MX Gene | Interferon, Newcastle | |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | Ela, SV40 Large T | Taylor et al., 1989; |
| | Antigen | Taylor and Kingston, |
| | | 1990a,b |
| Proliferin | Phorbol Ester-TPA | Mordacq and Linzer, |
| | | 1989 |
| Tumor Necrosis Factor | FMA | Hensel et al., 1989 |
| Thyroid Stimulating | Thyroid Hormone | Chatterjee et al., 1989 |
| Hormone α Gene | | |

TABLE 3
Other Promoter/Enhancer Elements

| Promoter/Enhancer | References |
|---|---|
| Immunoglobulin Heavy Chain | Hanerji et al., 1983; Gilles et al., 1983; |
| | Grosschedl and Baltimore, 1985; |
| | Atchinson and Perry, 1986, 1987; Imler |
| | et al., 1987; Weinberger et al., 1988; |
| | Kiledjian et al., 1988; Porton et al., 1990 |
| Immunoglobulin Light Chain | Queen and Baltimore, 1983; Picard and |
| | Schaffner, 1984 |
| T-Cell Receptor | Luria et al., 1987, Winoto and Baltimore, |
| | 1989; Redondo et al., 1990 |
| HLA DQ α and DQ β | Sullivan and Peterlin, 1987 |
| β-Interferon | Goodbourn et al., 1986; Fujita et al., 1987; |
| | Goodbourn and Maniatis, 1985 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRα | Sherman et al., 1989 |
| β-Actin | Kawamoto et al., 1988; Ng et al., 1989 |
| Muscle Creatine Kinase | Jaynes et al., 1988; Horlick and Benfield, |
| | 1989; Johnson et al., 1989a |
| Prealbumin (Transthyretin) | Costa et al., 1988 |
| Elastase I | Omitz et al., 1987 |
| Metallothionein | Karin et al., 1987; Culotta and Hamer, |
| | 1989 |
| Collagenase | Pinkert et al., 1987; Angel et al., 1987 |
| Albumin Gene | Pinkert et al., 1987, Tronche et al., 1989, |
| | 1990 |
| α-Fetoprotein | Godbout et al., 1988; Campere and |
| | Tilghman, 1989 |
| t-Globin | Bodine and Ley, 1987; Perez-Stable and |
| | Constantini, 1990 |
| β-Globin | Trudel and Constantini, 1987 |
| e-fos | Cohen et al., 1987 |
| c-HA-ras | Triesman, 1986; Deschamps et al., 1985 |
| Insulin | Edlund et al., 1985 |
| Neural Cell Adhesion | Hirsch et al., 1990 |
| Molecule (NCAM) | |
| a_{1-Antitrypain} | Latimer et al., 1990 |
| H2B (TH2B) Histone | Hwang et al., 1990 |
| Mouse or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid | Edbrooke et al., 1989 |
| A (SAA) | |
| Troponin I (TN I) | Yutzey et al., 1989 |
| Platelet-Derived Growth | Pech et al., 1989 |
| Factor | |
| Duchenne Muscular | Klamut et al., 1990 |
| Dystrophy | |
| SV40 | Banerji et al., 1981; Moreau et al., 1981; |
| | Sleigh and Lockett, 1985; Firak and |
| | Subramanian, 1986; Herr and Clarke, |
| | 1986; Imbra and Karin, 1986; Kadesch |
| | and Berg, 1986; Wang and Calame, 1986; |
| | Ondek et al., 1987; Kuhl et al., 1987 |
| | Schaffner et al., 1988 |
| Polyoma | Swartzendruber and Lehman, 1975; |
| | Vasseur et al., 1980; Katinka et al., |
| | 1980, 1981; Tyndell et al., 1981; |
| | Dandolo et al., 1983; deVilliers et |
| | al., 1984; Hen et al., 1986; Satake et al., |
| | 1988; Campbell and Villarreal, 1988 |
| Retroviruses | Kriegler and Botchan, 1982, 1983; |
| | Levinson et al., 1982; Kriegler et al., |
| | 1983, 1984a,b, 1988; Bosze et al., 1986; |
| | Miksicek et al., 1986; Celander and |
| | Haseltine, 1987; Thiesen et al., 1988; |
| | Celander et al., 1988; Chol et al., |
| | 1988; Reisman and Rotter, 1989 |
| Papilloma Virus | Campo et al., 1983; Lusky et al., 1983; |
| | Spandidos and Wilkie, 1983; Spalholz et |
| | al., 1985; Lusky and Botchan, 1986; |
| | Cripe et al., 1987; Gloss et al., 1987; |
| | Hirochika et al., 1987, Stephens and |
| | Hentschel, 1987; Glue et al., 1988 |
| Hepatitis B Virus | Bulla and Siddiqui, 1986; Jameel and |
| | Siddiqui, 1986; Shaul and Ben-Levy, |
| | 1987; Spandau and Lee, 1988 |
| Human Immunodeficiency | Muesing et al., 1987; Hauber and Cullan, |
| Virus | 1988; Jakobovits et al., 1988; Feng |
| | and Holland, 1988; Takebe et al., 1988; |
| | Rowen et al., 1988; Berkhout et al., 1989; |
| | Laspia et al., 1989; Sharp and Marciniak, |
| | 1989; Braddock et al., 1989 |
| Cytomegalovirus | Weber et al., 1984; Boshart et al., |
| | 1985; Foecking and Hofstetter, 1986 |
| Gibbon Ape Leukemia Virus | Holbrook et al., 1987; Quinn et al., 1989 |

Another signal that may prove useful is a polyadenylation signal (hGH, BGH, SV40).

The use of internal ribosome binding sites (IRES) elements are used to create multigene, or polycistronic, messages. IRES elements are able to bypass the ribosome scanning model of 5′-methylated cap-dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

As discussed above, in certain embodiments of the invention, a cell may be identified and selected in vitro or in vivo by including a marker in the expression construct. Such markers confer an identifiable change to the cell permitting easy identification of cells containing the expression construct. Usually, the inclusion of a drug selection marker aids in cloning and in the selection of transformants, for example, genes that confer resistance to neomycin, puromycin, hygromycin, DHFR, GPT, zeocin, tetracycline and histidinol are useful selectable markers. Alternatively, enzymes such as herpes simplex virus thymidine kinase (tk) or chloramphenicol acetyltransferase (CAT) may be employed.

The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV 40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between elements is flexible, so that promoter function is preserved when elements are inverted or moved relative to one another. In the tk promoter, the spacing between elements can be increased to 50 bp apart before activity begins to decline. Depending on the promoter, it appears that individual elements can function either co-operatively or independently to activate transcription.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Aside from this operational distinction, enhancers and promoters are very similar entities.

Promoters and enhancers have the same general function of activating transcription in the cell. They are often overlapping and contiguous, often seeming to have a very similar modular organization. Taken together, these considerations suggest that enhancers and promoters are homologous entities and that the transcriptional activator proteins bound to these sequences may interact with the cellular transcriptional machinery in fundamentally the same way.

In any event, it will be understood that promoters are DNA elements which when positioned functionally upstream of a gene leads to the expression of that gene. Most transgene constructs of the present invention are functionally positioned downstream of a promoter element.

D. Selectable and Screenable Markers

In certain embodiments of the invention, the cells containing the nucleic acid constructs of the present invention, may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Marker genes may also be utilized to assay for the presence a viral vector, and thus, to confirm infection and integration. The presence of a marker gene ensures the selection and growth of only those host cells which express the inserts. Typical selection genes encode proteins that confer resistance to antibiotics and other toxic substances, e.g., histidinol, puromycin, hygromycin, neomycin, methotrexate etc. and cell surface markers.

The inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of screenable markers such as GFP, whose basis is calorimetric analysis are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis.

Further examples of selectable and screenable markers are well known to one of skill in the art. The marker used is not believed to be important, so long as it is capable of being expressed simultaneously with the nucleic acid encoding a gene product, in this invention STAT-1α.

E. Cell Delivery and Pharmaceutical Compositions

In a preferred embodiment of the present invention, a method for preventing IL-1β- and IFN-γ-mediated cytotoxicity of a cell is contemplated. The preferred mode for in vivo delivery of a STAT-1α expression construct into a subject's cell is injection. Preferred ex vivo methods for preventing IL-1β- and IFN-γ-mediated cytotoxicity of cells in a subject, comprise a STAT-1α expression construct transferred into cell and administering the cell to the subject. In these embodiments, administration of the cell to the subject may be by injection, transplantation and the like.

1. Injection of STAT-1α Cells

Injection of cells comprising STAT-1α expression construct to a subject may be administered in combination with other agents as well, such as, e.g., various pharmaceutically-active agents. As long as the composition comprises at least one STAT-1α expression construct, there is virtually no limit to other components which may also be included, given that the additional agents do not cause a significant adverse effect upon contact with the cells.

The pharmaceutical cell compositions disclosed herein may be administered intradermally, parenterally, intravenously, intramuscularly, or even intraperitoneally as described in U.S. Pat. Nos. 5,543,158; 5,641,515 and 5,399,363 (each specifically incorporated herein by reference in its entirety). Injection of STAT-1α cell or cells may be delivered by syringe or any other method used for injection of a solution or suspension, as long as the cells can pass through the particular gauge of needle required for injection. A novel needeless injection system has recently been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

The formulation of pharmaceutically-acceptable excipients and carrier solutions are well-known to those of skill in the art, as is the development of suitable dosing and treatment regimens for using the particular compositions described herein in a variety of treatment regimens, including, e.g., intradermal, parenteral, intravenous, intramuscular, intranasal, and oral administration and formulation.

2. Cell Transplantation Methods

In general islet cell transplantation can be performed by several methods known to one of skill in the art. One of these methods, also known as the islet allograft model may be performed following published protocols (for example, see Gotoh et al., 1986). Briefly, donor pancreata are perfused in situ with type IV collagenase (2 mg/ml; Worthington Biochemical Corp., Freehold, N.J.). After a 40 minute digestion period at 37° C., the islets are isolated on a discontinuous Ficoll gradient. Subsequently, 300-400 islets are transplanted under the renal capsule of each recipient. Allograft function can be followed by serial blood glucose measurements (Accu-Check III™; Boehringer, Mannheim, Germany). Primary graft function is defined as a blood glucose level under 11.1 mmol/l on day 3 post-transplantation, and graft rejection is defined as a rise in blood glucose exceeding 16.5 mmol/l (on each of at least 2 successive days) following a period of primary graft function.

Common methods of administering pancreatic cells to subjects, particularly human subjects, include injection or implantation of the cells into target sites in the subjects, the cells of the invention can be inserted into a delivery device which facilitates introduction by, injection or implantation, of the cells into the subjects. Such delivery devices include tubes, e.g., catheters, for injecting cells and fluids into the body of a recipient subject. In a preferred embodiment, the tubes additionally have a needle, e.g., a syringe, through which the cells of the invention can be introduced into the subject at a desired location. The engineered cells of the invention can be inserted into such a delivery device, e.g., a syringe, in different forms. For example, the cells can be suspended in a solution or embedded in a support matrix when contained in such a delivery device. As used herein, the term “solution” includes a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by incorporating the engineered STAT-1α cells as described herein, in a pharmaceutically acceptable carrier or diluent and, as required, other ingredients enumerated above, followed by filtered sterilization.

Support matrices in which the engineered STAT-1α cells can be incorporated or embedded include matrices which are recipient-compatible and which degrade into products which are not harmful to the recipient. Natural and/or synthetic biodegradable matrices are examples of such matrices. Natural biodegradable matrices include plasma clots, e.g., derived from a mammal, and collagen matrices. Synthetic biodegradable matrices include synthetic polymers such as polyanhydrides, polyorthoesters, and polylactic acid. Other examples of synthetic polymers and methods of incorporating or embedding cells into these matrices are known in the art, for example, U.S. Pat. Nos. 4,298,002 and 5,308,701. These matrices provide support and protection for the fragile pancreatic cells in vivo and are, therefore, the preferred form in which the pancreatic cells are introduced into the recipient subjects.

Engineered cells may be implanted using the alginate-polylysine encapsulation technique of O'Shea and Sun (1986), with modifications as recently described by Fritschy, et al. (1991). The engineered cells are suspended in 1.3% sodium alginate and encapsulated by extrusion of drops of the cell/alginate suspension through a syringe into CaCl₂. After several washing steps, the droplets are suspended in polylysine and rewashed. The alginate within the capsules is then reliquified by suspension in 1 mM EGTA and then rewashed with Krebs balanced salt buffer. Each capsule should contain several hundred cells and have a diameter of approximately 1 mm.

Implantation of encapsulated islets into animal models of diabetes by the above method has been shown to significantly increase the period of normal glycemic control, by prolonging xenograft survival compared to unencapsulated islets (O'Shea, et al., 1986; Fritschy, et al., 1991). Also, encapsulation will prevent uncontrolled proliferation of clonal cells. Capsules containing cells are implanted (approximately 1,000-10,000/animal) intraperitoneally and blood samples taken daily for monitoring of blood glucose and insulin.

Recently, further methods for implanting islet tissue into mammals have been described (Lacy et al., 1991; Sullivan et al., 1991; each incorporated herein by reference). Firstly, Lacy and colleagues encapsulated rat islets in hollow acrylic fibers and immobilized these in alginate hydrogel. Following intraperitoneal transplantation of the encapsulated islets into diabetic mice, normoglycemia was reportedly restored. Similar results were also obtained using subcutaneous implants that had an appropriately constructed outer surface on the fibers. It is therefore contemplated that engineered cells of the present invention may also be straightforwardly “transplanted” into a mammal by similar subcutaneous injection.

The development of a biohybrid perfused “artificial pancreas”, which encapsulates islet tissue in a selectively permeable membrane, has also been reported (Sullivan et al., 1991). In these studies, a tubular semi-permeable membrane was coiled inside a protective housing to provide a compartment for the islet cells. Each end of the membrane was then connected to an arterial polytetrafluoroethylene (PTFE) graft that extended beyond the housing and joined the device to the vascular system as an arteriovenous shunt. The implantation of such a device containing islet allografts into pancreatectomized dogs was reported to result in the control of fasting glucose levels in 6/10 animals. Grafts of this type encapsulating engineered cells could also be used in accordance with the present invention.

In addition, various implantable therapeutical devices, such as drug delivery, gene therapy, and cell encapsulation devices, have also been developed which can be used to transplant the engineered insulinoma cells of the present invention. Most of these devices use selectively permeable, or semipermeable, membranes to construct all or part of the implantable device. These membranes contain their respective therapeutic agents and delivery systems within the particular device while being permeable to the desired therapeutical product. For cell encapsulation devices, the membranes are also permeable to life sustaining substances and to cellular waste products. Implanted cell encapsulation devices, particularly those intended as an artificial endocrine gland, usually require a high rate of flux of nutrients and waste products between the encapsulated cells in the device and tissues of the recipient. Having a cell encapsulation device in close, or direct, association with a vascular structure usually provides the highest rate of nutrient and waste product flux for such a device.

When certain therapeutical devices are implanted in a recipient, predominantly vascular tissues of the recipient can be stimulated to grow into direct, or near direct, contact with the device. This allows the therapeutical product of the device to be delivered directly to the circulation of the recipient through the vascular tissues that are in contact with the device. However, once vascular tissues of a recipient have grown in contact with one of these implantable therapeutical devices, removal of the device requires surgical dissection of the tissues to expose and remove the device. Surgical dissection of vascular tissues, particularly capillary tissue, can often be a difficult and painful procedure.

For cell encapsulation devices, an alternative to retrieving and replacing the entire device in a recipient is to retrieve and replace the cells contained in the device. U.S. Pat. No. 5,387,237, issued to Fournier et al., is a representative example of a cell encapsulation device that has at least one opening into the device through which cells can be introduced and removed. Cells are introduced and removed in this, and other similar devices, as a suspension or slurry. Since most cell encapsulation devices are intended to correct a metabolite deficiency in a recipient caused by dysfunction or failure of certain of the recipient's cells, tissues, or organs, the source of the replacement cells is rarely the recipient. In a situation where non-autologous cells are used in this type of cell encapsulation device, the problem of contaminating a recipient with the foreign cells during loading, removal, or refilling of the device is ever present. One solution to this contamination problem would be to enclose the cells in a container that can be placed, removed, and replaced in a device as a unit.

A retrievable cell encapsulation envelope enclosed in an implantable permselective membrane for use as an artificial endocrine gland is disclosed in U.S. Pat. No. 4,378,016 issued to Loeb. The Loeb device comprises a housing made of an impermeable hollow stem and a permselective membrane sack. The hollow stem has a distal end defining an extracorporeal segment, a percutaneous segment in the mid-region, and a proximal end defining a subcutaneous segment. The sack is adapted to receive an envelope containing hormone-producing cells and has an access opening that is coupled to the proximal end of the hollow stem. In a preferred embodiment, the cell containing envelope is in the form of a flexible collar. The flexible collar is partially collapsible to allow for easier placement and replacement of the envelope in the sack. Once in place, the flexible collar also provides a snug fit between the envelope and the sack. Placement and replacement of a cell containing envelope in the sack portion is accomplished manually with forceps, or the like. Retrieval of the envelope from the sack can be aided with a guidewire attached to the envelope. In one embodiment of the Loeb device, the sack has openings at both ends that are implanted percutaneously. In this embodiment, the cell containing envelope may be inserted or removed through either end of the device.

The housing of the Loeb device is surgically implanted in a recipient through the abdominal wall so the distal end of the stem protrudes from the recipient, the proximal end of the stem resides subcutaneously with respect to the abdominal wall, and the sack portion is placed in the peritoneal cavity surrounded in peritoneal fluid. According to Loeb, the sack allows hormones, nutrients, oxygen, and waste products to flow in and out of the sack while preventing bacteria from entering the patient. The sack and the envelope are said by Loeb to be permeable to nutrients and hormones, but impermeable to the hormone-producing cells and immune response bodies. Upon implantation of the device in a patient, the cells contained therein are said to take over the function of the corresponding natural gland, sense the amount of hormone needed, and produce the correct amount of the desired hormone.

Brauker et al. disclose a cell encapsulation device in U.S. Pat. No. 5,314,471 that requires close association of host vascular structures with the device. Cell death in these implanted devices is said by Brauker et al. to be due in large part to an ischemia imposed on the cells during the first two weeks following implantation. The development of new vascular structures close to the cell encapsulation device can be provided by angiogenic factors applied to the cell boundary of the device.

In U.S. Pat. No. 5,843,069, incorporated herein by reference, Butler et al., report an implantable containment apparatus, made of selectively permeable materials used to contain a therapeutical device, such as a drug delivery device, a cell encapsulation device, or a gene therapy device. A therapeutical device can be easily placed and replaced in this apparatus without damaging tissues associated with the selectively permeable material of the apparatus. The patent describes how the selectively permeable material permits exchange of solutes between a therapeutical device contained in the apparatus and tissues of a recipient, while excluding cells from growing beyond a desired point through the material. The patent also describes how upon implantation of the apparatus in a recipient, various tissues from the recipient grow to associate with the apparatus. These tissues grow next to, or partially through, the exterior surface of the apparatus. Growth of vascular tissue as the predominant tissue in association with the apparatus removes the need to supply angiogenic factors and allows the placement and replacement of any therapeutical device without damaging or disturbing the tissues associated with the selectively permeable material of the apparatus. U.S. Pat. No. 5,913,998, also incorporated herein by reference, describes further embodiments of the apparatus and devices described.

Cell encapsulation devices, such as those described in U.S. Pat. No. 5,902,745 to Butler, et al, incorporated herein by reference, may also be used for cell transplantation in the present invention. The cell encapsulation device, of U.S. Pat. No. 5,902,745, permits rapid and straightforward cell transfer. The features of that device are components that provide: automatic filtration of excess solution during cell transfer; an instantly wettable cover, allowing ready view into the cell chamber; and a swellable core, allowing cells to be transferred with minimal shear force while assuring optimal cell placement in the device during use. The device described in that invention can be used either in vivo, such as to deliver therapeutic substances, or in vitro, such as to serve as a bioreactor. The device is particularly suitable for containing genetically engineered cells while permitting a desired gene product produced by the encapsulated cells to be delivered from the cells to a patient or a tissue culture.

In U.S. Pat. No. 5,980,889, incorporated herein by reference, Butler, et al., further describe, cell encapsulating devices capable of maintaining large numbers of viable cells containing an inert, substantially cell-free core that displaces cells, a permeable membrane and a zone for maintaining cells. The permeable membrane surrounds the core such that the zone of cells is bounded by the core and the permeable membrane. The cell zone may contain a support means for cell attachment and the core may have an outer boundary containing a material that promotes cell adhesion

Hayes, et al., (U.S. Pat. No. 6,031,148), incorporated herein by reference, describe a method for making an implantable bioabsorbable article which can be used for the separation and regeneration of a tissue at a tissue defect site in the form of a fibrous matrix laminarly affixed to both surfaces of a cell-barrier sheet material. When this article is implanted it allows ingrowth of tissue into the fibrous matrix side of the material; simultaneously allowing the separation of tissue to be regenerated at the tissue defect site from the ingrowing tissue by the cell-barrier sheet material. The cell-barrier sheet material and the fibrous matrix can be composed of synthetic bioabsorbable materials such as polylactic acid, polyglycolic acid, poly-P-dioxanone, trimethylene carbonate, polycaprolactone and copolymers thereof.

The devices described have several components which may be constructed from any suitable biocompatible material, including polytetrafluoroethylene, poly(dimethyl siloxane) rubber, polyurethane, polyethylene, polyethylene vinyl acetate, polypropylene, polybutadiene, polyvinyl chloride, polyvinyl acetate, polyacrylonitrile, polyamide, glass or glass fibers, stainless steel, and other materials known to those skilled in the art. Other materials include swellable polymers which may be selected from the group consisting of hydrophilic polyacylonitrile such as HYPAN.RTM. Structural Hydrogel (Hymedix International, Inc., Dayton, N.J.), chitin, chitosan, hydroxyethylmethacrylate (HEMA), hydrophilic polyurethane, polyethylene glycol, polyacrylamide, polyacrylic acid, and silica gel, or hydrogels derived from polysaccharides, such as alginate and other materials known to those skilled in the art. Yet, other components of the devices may be made from flexible polymer or elastomer or blends with copolymers of swellable and non-swellable material including but not limited to polysaccharides, hydrophilic copolymers of polyacrylonitrile, or other polymer components. The selectively permeable components of the transplantable devices are generally made of microporous material which include, but are not limited to: expanded polytetrafluoroethylene (ePTFE); woven/and or non-woven polymers such as polyester, polyethylene terephthalate, polyamide, vinyl acetates, polypropylene, polyethylene, polyacrylonitrile, polyaramide, polyhydroxy acids (such as polylactic acid, polyglycolic acid, or polycaprolactone); open cellular foams such as phenolic and epoxy resins, polyurethanes, chloroprene, isoprene, polyethylene, polypropylene, polystyrene, polyvinyl chloride, latex foam rubber, polyurea-formaldehyde, polyhydroxy acids (such as polylactic acid, polyglycolic acid, polyhydroxybutyric acid, polyhydroxyvaleric acid, or polycapralactone); and poromerics and/or permeable matrix membranes (such as polyvinyls, polyamide, polyimide, polyacrylonitrile, polysulfone, polyvinylidene fluoride, polypropylene, polymethylmethacrylate, polycarbonate, regenerated cellulose, or cellulose acetate).

The cell implantable and or transplantable devices described enable cell encapsulation, cell insertion, cell retrieval, and cell replacement. Thus, these transplantable devices can be used in the present invention to achieve the transplantation of insulinoma cells and or any other cells which have been genetically engineered to avoid cellular cytotoxic destruction.

F. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Material and Methods

Cells and reagents. The rat insulinoma cell line INS I was cultured in RPMI 1640 medium containing 10% fetal calf serum, 10 mM Hepes, 2 mM L-glutamine, 1 mM Napyruvate, 50 uM β-mercaptoethanol, 100 U/ml of penicillin, and 100 ug/ml streptomycin (Sigma Chemical Co., St. Louis, Mo.) at 37° C. and 5% CO₂. Recombinant rat IL-1β was obtained from Endogen (Cambridge, Mass.). Recombinant rat IFN-γ was obtained from GIBCO BRL (Gaithersburg, Md.). L-NMMA (Sigma) was prepared as a 100 mM solution in PBS and diluted to a final concentration of 1 mM in culture medium.

Selection of resistant cell line. The selection process was initiated with INS-1 cells in 30 mm culture dishes containing culture medium supplemented with 0.5 ng/ml of rat IL-1β and 5 U/ml of rat IFN-γ. The culture medium was changed every 3 days. Surviving cells were trypsinized, harvested, pooled, and recultured at 0.5 ng/ml IL-1β and 5 U/ml rat IFN-γ. After the cells started to grow, the concentration of cytokines was increased to 1 ng/ml of rat IL-1β and 10 U/ml of rat IFN-γ. The process was repeated at increasing concentration of cytokines (2.5, 5, 10 ng/ml of rat IL-1β and 25, 50, 10 U/ml, of rat IFN-γ) until the concentration of rat IL-1β and rat IFN-γ reached 10 ng/ml and 100 U/ml, respectively. Cells that were recovered after the entire selection protocol were termed INS-1ᵣₑₛ. In some experiments, INS-1ᵣₑₛ cells were allowed to grow in medium containing 10 ng/ml of rat IL-1β and 100 U/ml of rat IFN-γ for about 1 week, and then switched to medium lacking cytokines for a further 6 week culture period. To assess the stability of the resistant phenotype, clonal lines were derived by stable transfection of parental INS-1 cells or INS-1ᵣₑₛ cells with a plasmid containing the cDNA encoding human proinsulin.

MTT viability assay. The MTT viability assay has been described previously (Mossman, T., 1983) and (Schnedl et al., 1994). Briefly, cells were trypsinized, counted and inoculated at 80,000 cells/well in flat-bottom 96 well tissue culture plates. After 24 hours in RPMI culture medium lacking cytokines, replacement medium containing various concentrations of cytokines was added as specified in the brief description of the drawings. After 48 hours, the test media were discarded and replaced by 115 μl/well of medium with 75 μg/ml of C,N diphenyl-N′-[4,5-dimethylthiazol-2-yl]-2,5-diphenyltetrazolium bromide (MTT) for 1.5 hour at 37° C. and 5% CO₂. The resulting formazan crystals were solubilized in 115 ul 0.04N HCl in isopropanol. The optical density of the solubilized formazan was read at 575 and 650 nm using a SpectraMax 340 (Molecular Devices, Sunnyvale, Calif.) plate reader. The reduction in optical density caused by cytokine treatment was used as a measurement of cell viability and normalized to cells incubated in control medium which were considered 100% viable.

Preparation of activated supernatants from rat PBMCs. Heparinized blood was collected from Wistar rats (Charles River Breeding Laboratories), and rat peripheral blood mononuclear cells (PBMC) were isolated over Histopaque 1077 (Sigma Chemical Co., St. Louis, Mo.) using standard methods. 2×10⁶ PBMC/ml were incubated in RPMI 1640 medium supplemented with 10% fetal calf serum, 100 U/ml of penicillin, and 100 μg/ml streptomycin. PBMC were stimulated for 3 days with 10 μg/ml LPS (Escherichia coli 0127:B8, Sigma), 10 ng/ml of PMA (Sigma), or 10 μg/ml of Concanavalin A (Sigma) in the culture medium. The conditioned media were harvested and centrifuged at 2,000 g for 15 minutes to remove cellular components. The supernatants were kept at −20° C. Before adding the conditioned media to the cells, an appropriate volume of 50×INS-1 supplement (0.5 M Hepes, 100 mM L-glutamine, 50 mM Na-pyruvate, 2.5 mM β-mercaptoethanol, pH 7.4) was added to make the conditioned media contain the same supplements as normal INS-1 medium.

Insulin Secretion Experiments: INS-1 or INS1ᵣₑₛ cells were cultured in 12 well plates. To initiate insulin secretion assays, cell culture medium was removed, cells were washed with 1 ml of PBS (Life Technologies, Inc.) and then with 1 ml of secretion assay buffer (SAB), containing 3 mM glucose. SAB contains 114 mM NaCl, 4.7 mM KCl, 1.2 mM KH2PO4, 1.16 mM MgSO4, 2.5 CaCl 2, 25 mM NaHCO3, 20 mM HEPES, and 4% fatty acid-free bovine albumin, pH, 7.4. After the wash, cells were incubated with 0.5 ml of SAB containing 3mM glucose for 2 hours at 37° C. This media was collected and replaced with 0.5 ml of SAB containing 15 mM glucose for another 2 hours. All media samples were centrifuged at 3000 rpm for 6 minutes to remove the floating cells. The supernatants were subjected to insulin radioimmunoassay, using a human insulin standard (DPC Coat-A-Count). The cells remaining in the wells were then washed once with PBS and lysed with 200 μl of PBS containing 0.5% triton X-100. Total protein levels were measured by the Bradford method (15), using a kit from Bio-Rad and bovine serum albumin as standard (Bio-Rad Laboratories, Hercules, Calif.).

RNA isolation and analysis. Total RNA was isolated from cells by TRIzol (GIBCO BRL Grand island, N.Y.) according to the manufacturer's protocol. 10 μg of total RNA was resolved on a 1.5% formadehyde agarose gel, and samples were transferred to nylon membrane and hybridized with ³²P-labeled cDNA probes using Rapid-Hyb buffer (Amercham Life Science) in a Hybaid Micro 4 Hybridization Oven (National Labnet Company). The radiolabeled probes were prepared from the cDNAs encoding the interleukin-1 receptor type I (IL-1RI) or the γ-IFN receptor (γ-IFNR) (provided by Dr. Anice Thigpen; BetaGene, Inc., Dallas, Tex.). Xbal fragments of the cDNAs encoding IL-RI or γ-IFNR were isolated by gel purification using the DNA-purification kit (QIAGENE and radiolabeled with ³²P-labeled dCTP by random priming with the redi-prime labeling kit (Amersham Life Science, Arlington Height, Ill.). After hybridization and washing, nylon membranes were exposed to films to create autoradiographs. Signals were quantified by Phosphoimager (Molecular Dynamic).

### EXAMPLE 2

### Selection of an INS-1 Cell Population Resistant to Cytokine-mediated Cytotoxicity

It has been demonstrated that the inflammatory cytokines IL-1β and IFN-γ induce cytotoxicity in INS-1 cells (Mandrup-Poulsen, T., 1996), (Rabinovitch, A., 1993), (Corbett and McDaniel, 1992), (Eizirik et al., 1996) (Hohmeier et al., 1998) and (Hakan et al., 1992). In the current study, the inventors tested a selection strategy designed for obtaining INS-1 cells that are resistant to both of these cytokines. To this end, INS-1 cells were cultured in medium with increasing concentrations of IL-1β+IFN-γ over an 8 week period, beginning at 0.5 ng/ml+5 IU/ml, and ending at 10 ng/ml+100 IU/ml, respectively. Cells carried through this entire procedure grew well at the highest cytokine concentrations, and were designated INS-1ᵣₑₛ.

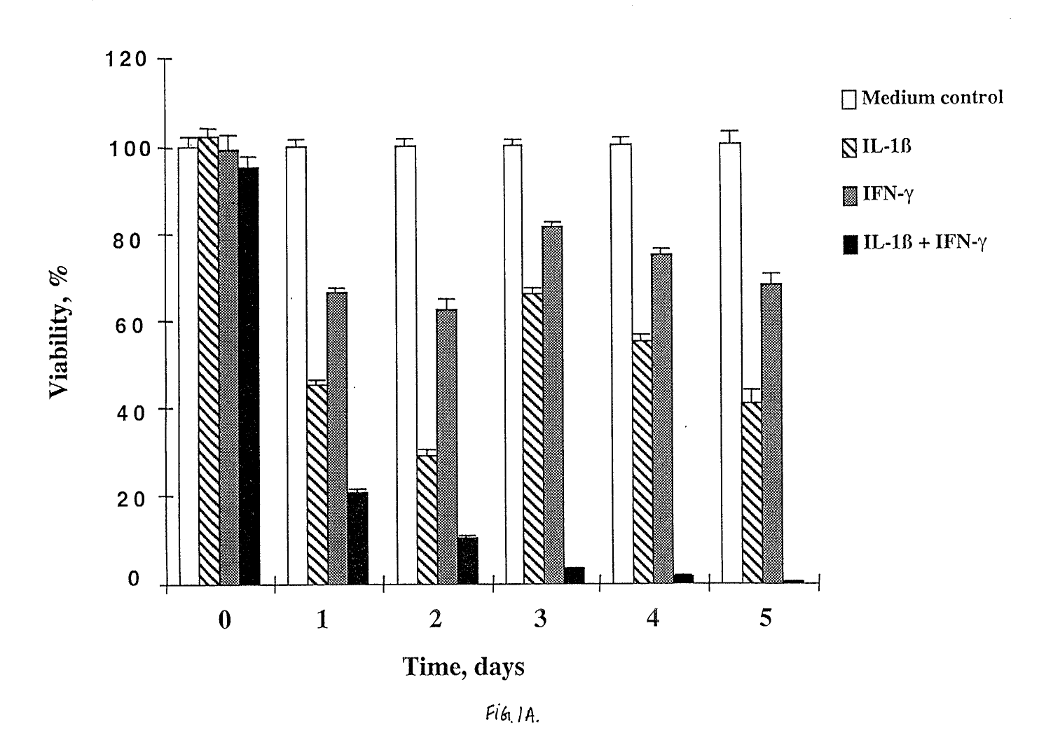

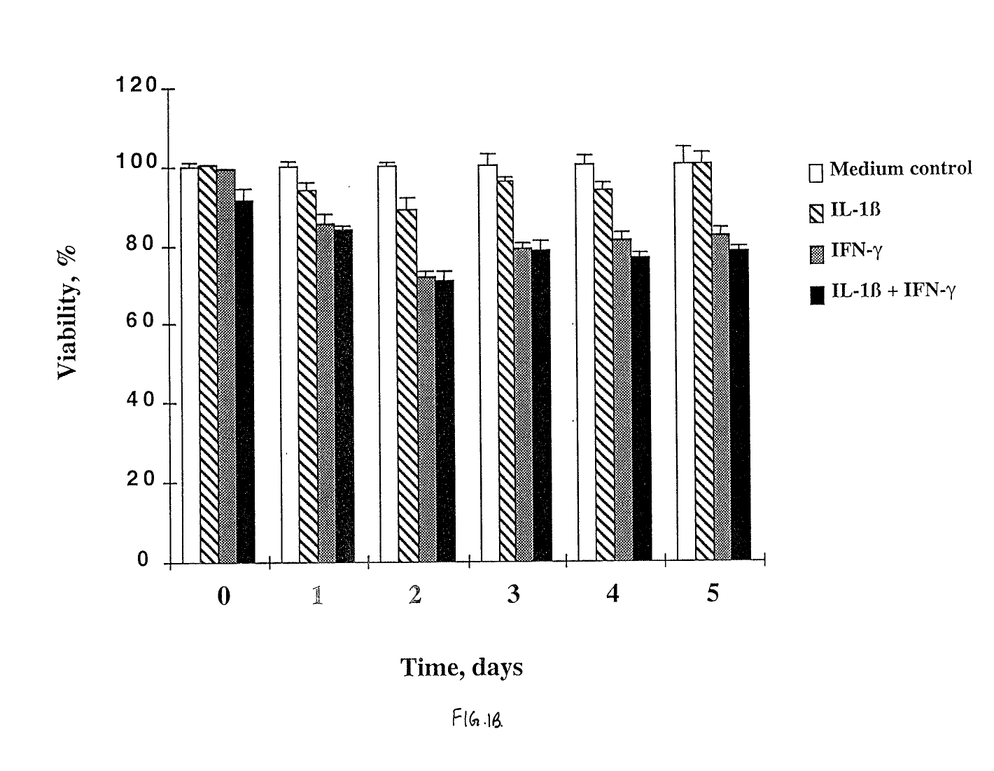

The degree of cytokine resistance achieved by this selection strategy was investigated using an MTT cell viability assay. Parental INS-1 and INS-1ᵣₑₛ cells were treated with IL1-β (10 ng/ml), IFN-γ (100 U/ml) or IL-1β (10 ng/ml)+IFN-γ (100 IU/ml). In parental INS-1 cells, 1 day of treatment with IL-1β or IFN-γ alone reduced viability to 40±3% and 68±3%, respectively, relative to untreated cells, with similar viabilities at longer time periods of treatment with these cytokines (FIG. 1A). The combination of IL-1β (10 ng/ml)+IFN-γ (100 U/ml was much more potent, causing a sharp drop to 21±0.4% viability after 1 day of exposure, and then a continued decline to near-complete cell destruction (0.4±0.03% viability) after 5 days. In sharp contrast, cell viabilities of INS-1ᵣₑₛ cells were maintained at 89±1.3%, 72 ±1.1%, and 71±2.6% at 2 days, and 100±1%, 82±1%, and 78±1% after 5 days of treatment with IL-1β, IFN-γ, or both cytokines, respectively (FIG. 1B). These results confirm that INS-1ᵣₑₛ cells have gained resistance to cytokine-induced cell damage.

### EXAMPLE 3

### INS-1ᵣₑₛ Cells are Resistant to the Cytotoxic Effects of Conditioned Medium From Activated PBMC

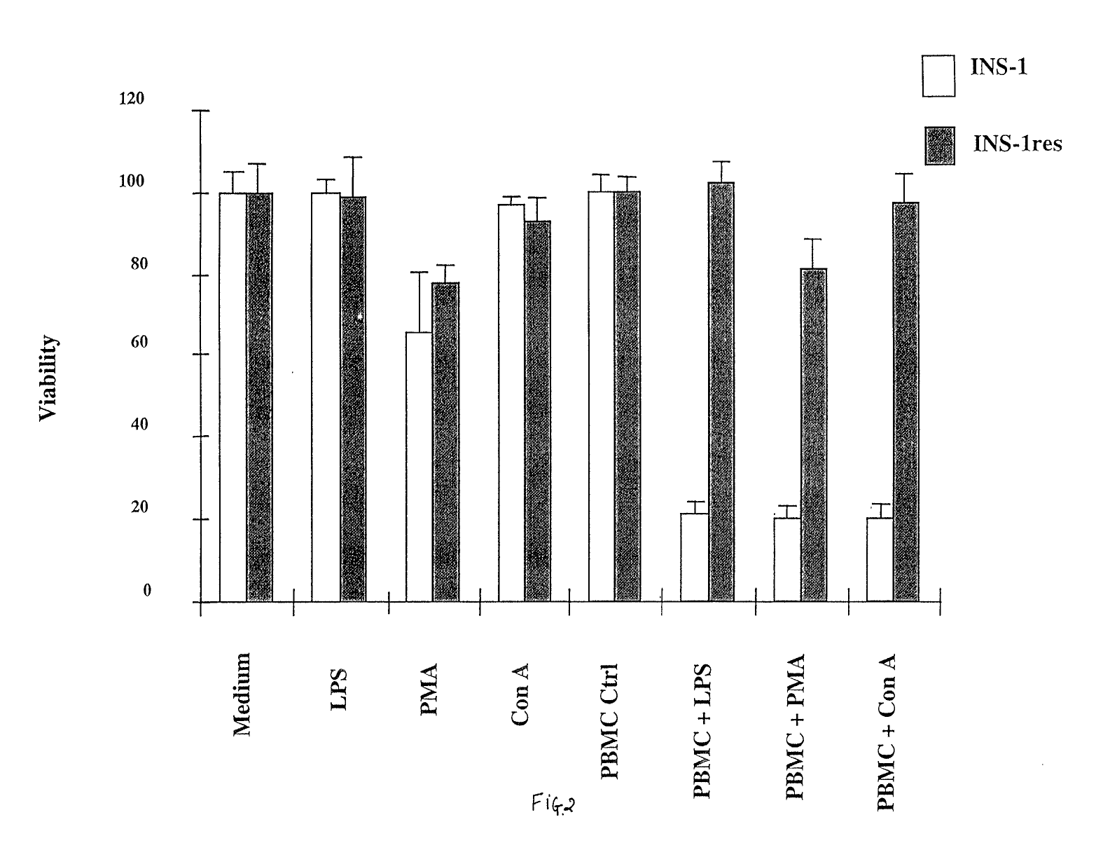

It was determined further whether the resistance of INS-1ᵣₑₛ cells to cytokines can be extended to killing mediated by a complex mixture of cytokines and other toxins. Conditioned media was collected from rat PBMC stimulated by LPS, PMA, or Con A for 72 h, and applied to parental INS-1 or INS-1ᵣₑₛ cells for 48 h. As shown in FIG. 2, cell viability was improved form 21±3%, 20±2% and 20±2% in INS-1 cells to 102±4%, 81±7% and 97±7% in INS-1ᵣₑₛ cells following incubation in media from LPS, PMA, or ConA-treated PBMC, respectively. Thus INS-1ᵣₑₛ cells gain almost complete resistance to the effects of conditioned medium from activated rat PBMC.

### EXAMPLE 4

### Relative Stability of the Resistant Phenotype in INS-1ᵣₑₛ Cells

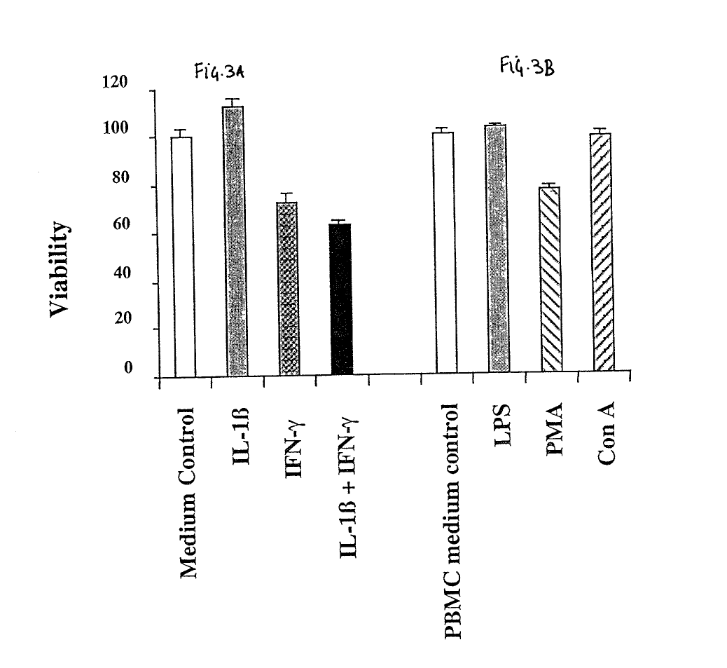

The stability of the resistant phenotype in INS-1ᵣₑₛ cells was investigated by growth of these cells in the absence of cytokines for 6 weeks. After this period, INS-1ᵣₑₛ cells still retained 113±3%, 73±3%, and 63±2% viability upon 48 h of exposure to IL-1β, IFN-γ, or IL-1β+IFN-γ, respectively (FIG. 3A). Similar studies were performed with conditioned media from rat PBMC, revealing viabilities of 103±3%, 77±5%, or 99±5% for INS-1ᵣₑₛ cells treated with media prepared form LPS, PMA or Con A-treated PBMC, respectively (FIG. 3B). These results indicated that resistance to IL-1β induced killing is a stable feature of INS-1ᵣₑₛ cells, while resistance to killing induced by IFN-γ requires continued presence of the cytokine in the tissue culture medium.

### EXAMPLE 5

### Expression of IL-1 type I and IFN-γ Receptors in INS-1ᵣₑₛ Cells

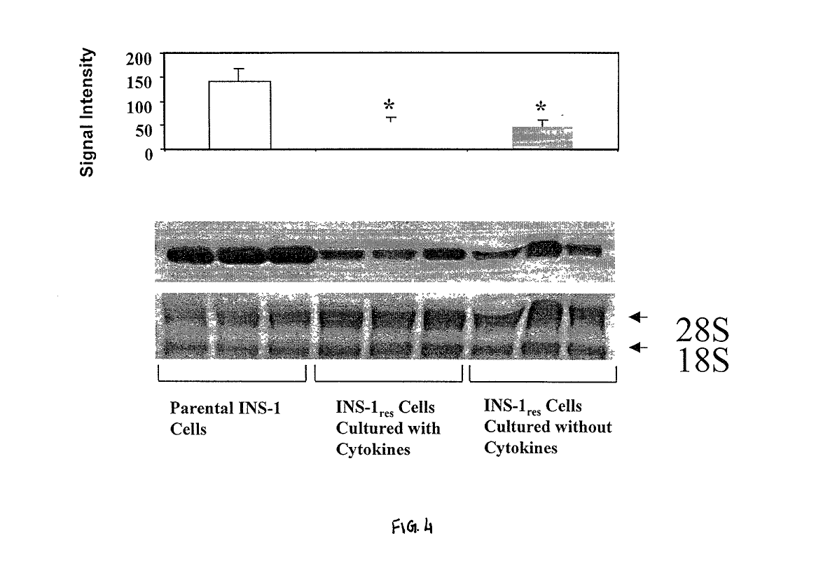

To investigate the mechanism of cytokine resistance in INS-1 ᵣₑₛ cells, the levels of mRNA encoding the IL-1 type 1 receptor (IL-1RI) and the IFN-γ receptor were compared in parental INS-1 cells, INS-1ᵣₑₛ cells kept in normal culture medium, and INS-1ᵣₑₛ cells kept in culture medium containing IL-1β (10 ng/ml)+IFN-γ (100 units/ml). As shown in FIG. 4, INS-1ᵣₑₛ cells exhibited a 60% decrease in IL-1RI mRNA levels compared to parental INS-1 cells. This reduction was stable after the withdrawal of the cytokines from the culture medium for more than 4 months. In contrast, levels of IFN-γ receptor mRNA were found to be unchanged among the three groups of cells (FIG. 4).

### EXAMPLE 6

### INS-1ᵣₑₛ Cells Transfected with the Human Insulin Gene and Selected in the Absence of Cytokines Retain Their Resistance to Cytokine-mediated Killing

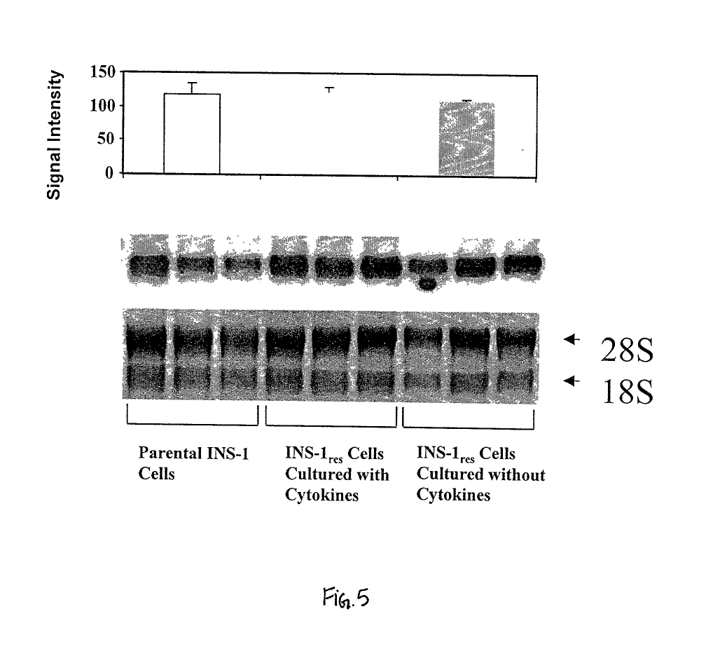

Stable transfection of INS-1ᵣₑₛ cells with the human insulin gene and selection of clones was carried out in the absence of cytokines in the growth medium (occupying a period of approximately 2 months). To investigate whether INS-1ᵣₑₛ cells retained their resistance to cytokine-mediated killing after this process, the viability of all of the INS-1ᵣₑₛ cells retained their resistance to cytokine-mediated killing after this process, the viability of all of the INS-1 and INS-1ᵣₑₛ clones (58 and 51 clones, respectively) was compared by MTT assay. As shown in FIG. 5, the average viability of cells treated with the combination of IL-1β and IFN-γ for 48 hours was 15±7% for clones derived form parental INS-1 cells, compared (to 53±11%) for clones derived from INS-1ᵣₑₛ cells. These results demonstrate that significant cytokine resistance is retained in the INS-1ᵣₑₛ population even after a round of stable genetic engineering.

### EXAMPLE 7

### INS-1ᵣₑₛ Clones Exhibit Permanent IL-1β Resistance, but Reversible IFN-γ Resistance

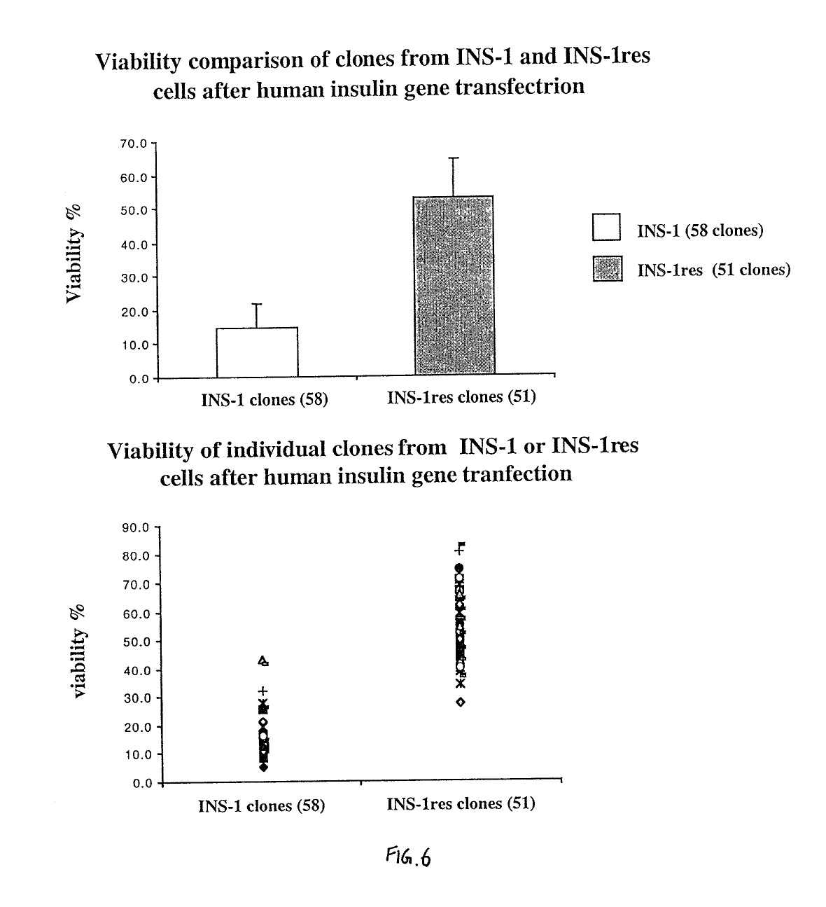

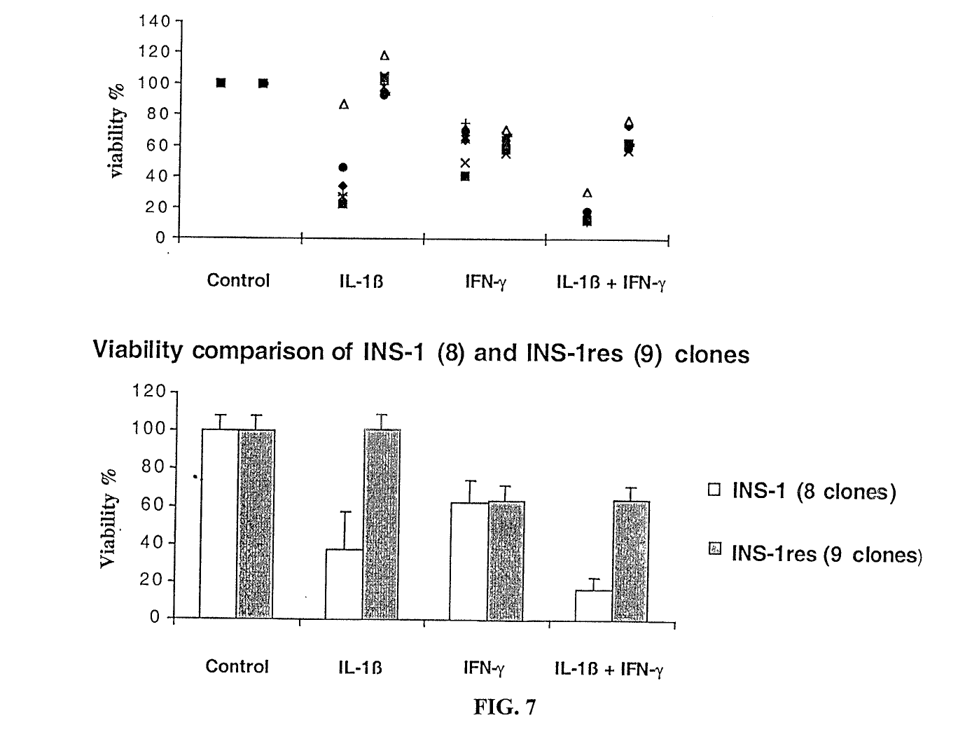

Clones derived from INS-1ᵣₑₛ cells by stable transfection were on average less resistant to the combination of IL-1β+IFN-γ (53% viability) than the starting INS-1ᵣₑₛ population (78% viability). To determine whether this is due to loss of resistance to one or both cytokines, viability assays were performed on the 8 glucose responsive INS-1 clones and 9 glucose responsive INS-1ᵣₑₛ clones treatment with IL-1β, IFN-γ, or the combination of IL-1β+IFNγ. The 9 INS-1ᵣₑₛ were all 100% resistant to IL-1β-mediated cytotoxicity, but were only 64±6.7% and 64±7.2% viable after treatment with IFN-γ or IFN-γ+1L-1β for 48 h (FIG. 6). Taken together, these data strongly suggest that cytokine-mediated killing of the INS-1ᵣₑₛ clones is attributable to IFN-γ. In contrast to these findings, clones derived from parental INS-1 cells were only 37±20%, 63±11% and 16±6.4% viable following treatment with IL-1β, IFN-γ, or IL-1+IFN-γ, respectively (FIG. 6).

To determine whether resistance to IFN-γ could be reinstated in insulin-transfected clones derived from INS-1ᵣₑₛ cells, all 9 clones were culture in medium containing IL-1β+IFN-γ for two weeks. Following this culture period, the transfected INS-1ᵣₑₛ clones were found to have average viabilities of 95±3.7%, 80±4.4% and 81±10% after culture in IL-1β, IFN-γ, or IL-1β+IFN-γ, respectively. This shows that the resistance of INS-1ᵣₑₛ clones to IFN-γ-mediated damage is reversible and reinducible in response to relatively short periods of culture in the presence and absence of cytokines, while resistance to IL-1β is unaffected by these maneuvers.

### EXAMPLE 8

### Stat-1α Expression is Inversely Related to Immunocytoxicity

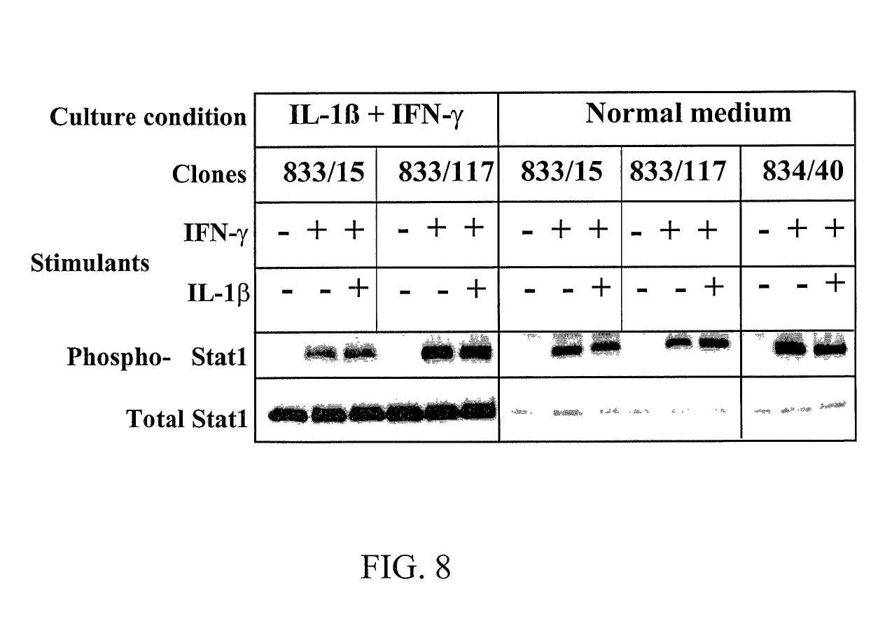

The inventors explored the molecular basis of cellular resistance to cytokine-mediated cytotoxicity. INS-1-derived (833/40) and INS-1ᵣₑₛ-derived (833/15, 833/117) clonal cell lines were grown in the presence or absence of IL-1β+IFN-γ. The results shows that cytokine resistance (achieved in the INSᵣₑₛ-derived lines when cultured in cytokines) is correlated with a large increase in expression of STAT-1α. FIG. 8.

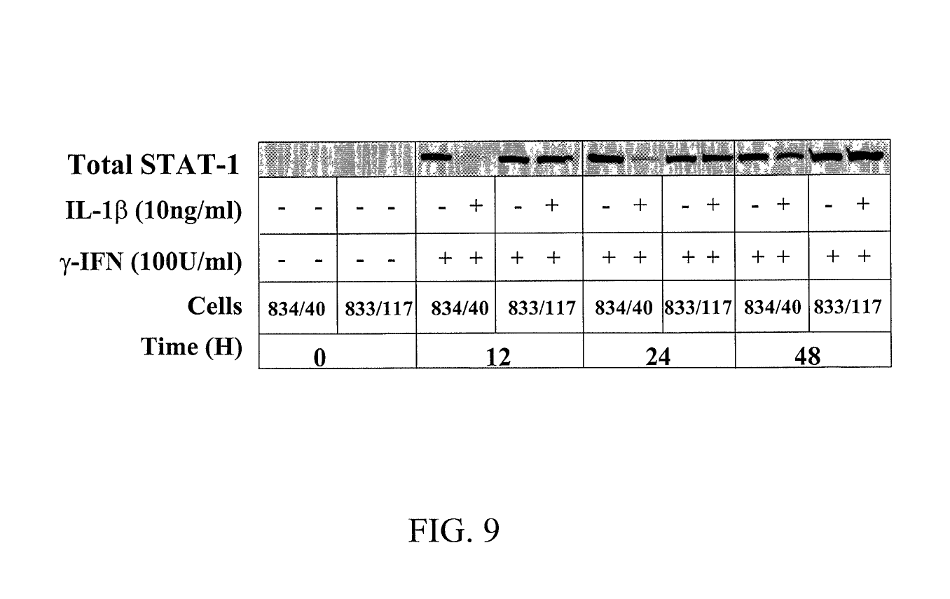

INS-1-derived (834/40) and INS-1ᵣₑₛ-derived (833/117) cells were cultured in normal medium lacking cytokines for a period of 6 months. Cells were then treated with IFN-γ or IFN-γ+IL-1β. In the cytokine-sensitive line, induction of STAT-1α expression by cytokines is suppressed by normal IL-1β signaling, rendering these cells vulnerable to IFN-γ-mediated cytotoxicity. The resistant cells, in contrast, have permanent resistance to IL-1β due to the selection procedure used for their derivation, thereby alleviating suppression of STAT-1α induction by this cytokine. FIG. 9.

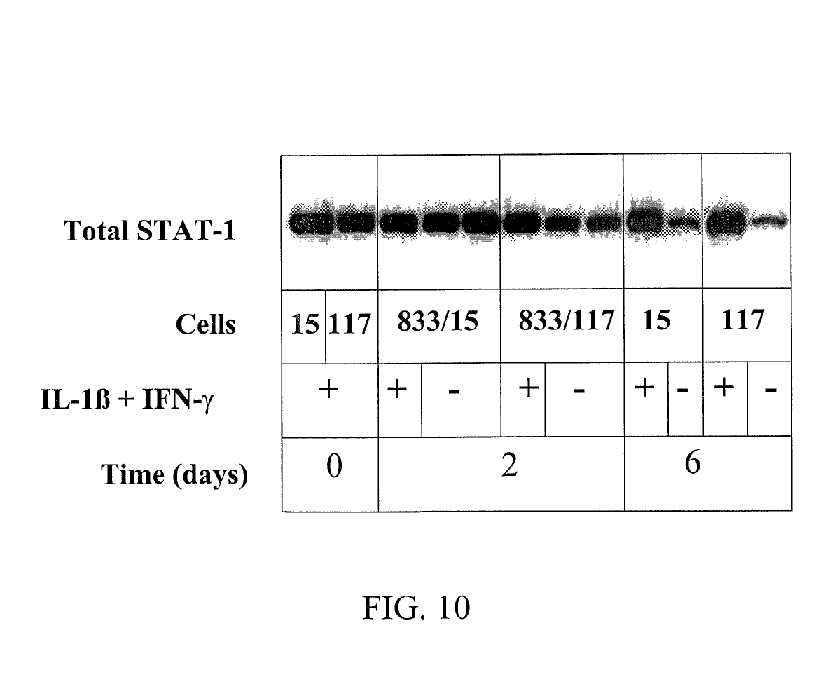

In order to confirm the importance of selective pressure on STAT-1, INS-1ᵣₑₛ clones (833/15 and 833/117) were inoculated into 12 well plates in the presence of IL-1β+IFN-γ. The data show that STAT-1α expression is decreased when cytokines are withdrawn from resistant cell lines, consistent with the return of IFN-γ and IFN-γ-+IL-1β-mediated cytotoxicity, providing further support for a protective role of elevated STAT-1α expression. FIG. 10.

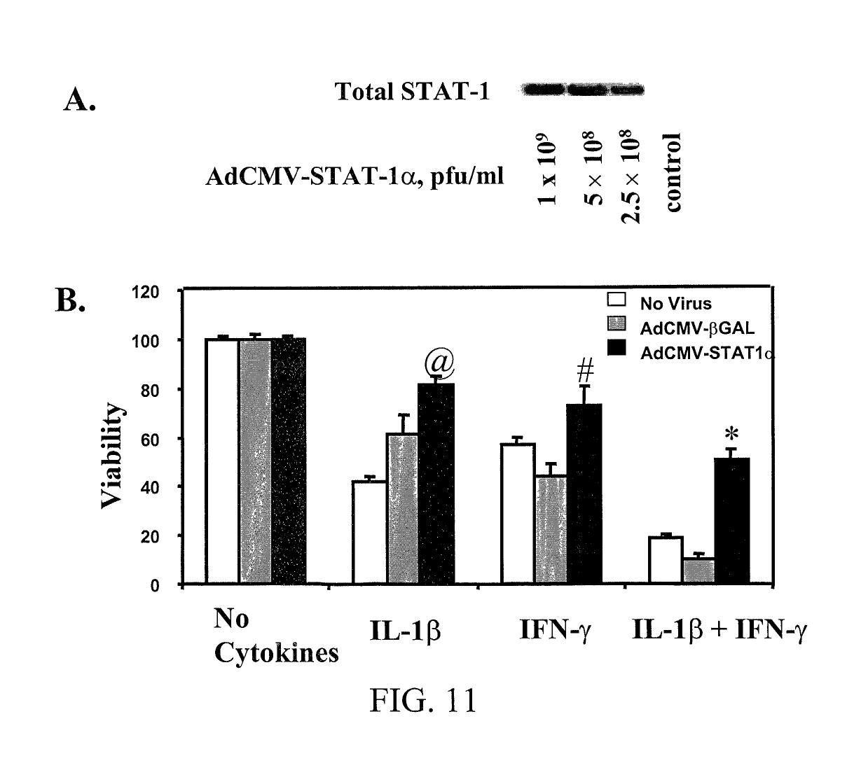

To confirm the role of STAT-1 on protection, 2.5×10⁴ cells were inoculated in 96 well plates overnight and treated with adenoviruses containing genes encoding β-galactosidase (βGAL) or STAT-1α (Ad.STAT1), or were left untreated (Medium). Note that STAT-1α expression significantly reduces the cytotoxic effects of IL-1β+IFN-γ relative to either control group (* signifies that Ad/Stat1 groups is different from medium and β-gal control groups, with p=0.016 and 0.0025, respectively. FIG. 11B.

### EXAMPLE 9

### Adenovirus-mediated Overexpression of STAT1α Reduces NO Production and iNOS Expression

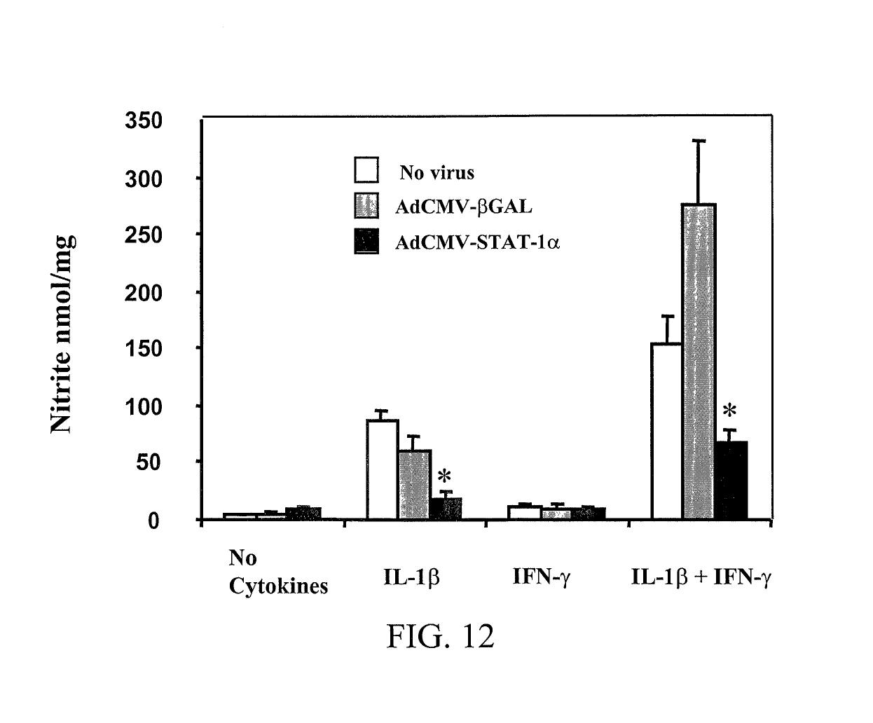

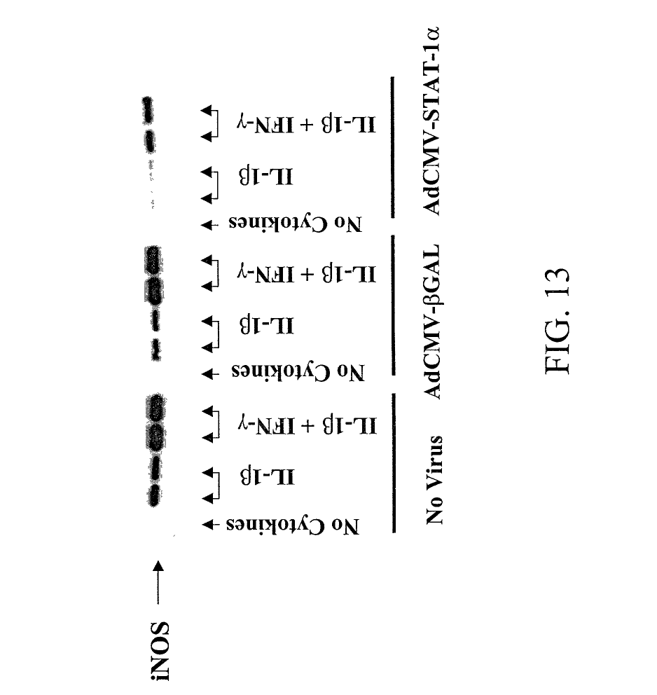

IL-1β is known to stimulate NO production in islet β-cells via induction of iNOS, and NO production has been implicated in IL-1β-mediated β-cell destruction (Mandrup-Poulsen, 1996); Rabinovitch, 1993; Corbett, et al. 1992; Eizirik, et al. 1996; Hohmeier, et al. 1998). Inventors have shown that the iNOS inhibitor, L-NMMA can block cytotoxicity mediated by IL-1β, and that INS-1ᵣₑₛ cells do not produce NO in response to acute treatment with IFN-γ, IL-1β, or IL-1β+IFN-γ (Chen, et al. 2000; Hohmeier, et al. 1998). To test whether STAT-1α overexpression affects NO production and iNOS expression in cytokine sensitive 834/40 cells, cells were treated as described in FIG. 12 with AdCMV-STAT-1α, AdCMV-βGAL, or no virus, and were then exposed to 10 ng/ml IL-1β, 100 U/ml IFN-γ, or the combination of IL-1β+IFN-γ for 48 h. As shown in FIG. 12, after 48 h of treatment with IL-1β or I1β+IFN-γ, nitrite production (a measure of NO accumulation) was significantly reduced in STAT-1α overexpressing 834/40 cells compared with either control group. The impairment in NO production in STAT-1α expressing cells was already apparent within 9 h of cytokine addition, proving that expression of the transcription factor effectively blocks the acute response to cytokine treatment. Consistent with these findings, AdCMV-STAT-1α-treated cells exhibited a 70% reduction in IL-1β-induced accumulation of iNOS protein relative to untreated or AdCMV-βGAL-treated control cells, and a 50% reduction following treatment with IL-1β+IFN-γ, based on densitometric scanning of the two representative immunoblots shown in FIG. 13. Taken together, these data suggest that protection against cytokine-mediated cytotoxicity in STAT1α overexpressing cells is in part attributable to reduced NO production and iNOS expression. It is interesting to note that the impairment of cytokine-induced NO production in STAT-1α overexpressing cells is partial, in contrast to findings in cells taken through the cytokine selection process, in which NO production was completely blocked (Chen, et al. 2000). These results imply that factors other than STAT-1α expression, possibly including impairment of IL-1β-induced Nf-kB translocation (Chen, et al. 2000), contribute to the more complete blockade of NO production noted in the INS-1ᵣₑₛ cell lines.

### EXAMPLE 10

### Adenovirus-mediated Overexpression of STAT1α Does not Impair Robust Glucose-stimulated Insulin Secretion in INS-1-derived Cell Lines

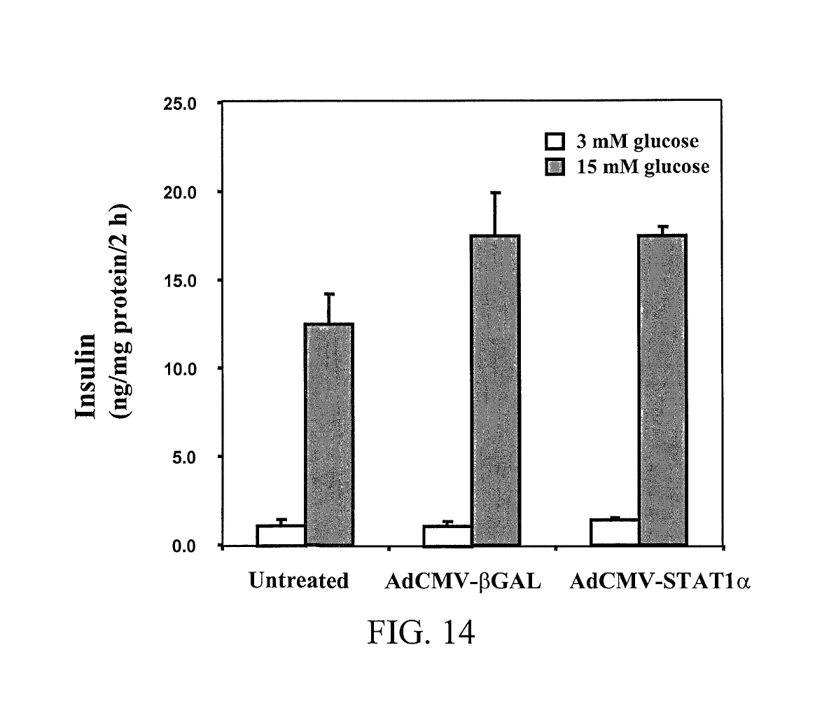

For STAT-1α expression is to serve as a viable strategy for protecting insulin-producing cells against cytokine-mediated damage, it is important to demonstrate that expression of this gene does not impair glucose-stimulated insulin secretion. The inventors have recently demonstrated that the rat insulinoma cell line INS-1 (Asfari, et al. 1992) is comprised of a mixture of glucose responsive and unresponsive cells (Chen, G. et al. 2000; Hohmeier, et al. 2000). Inventors therefore tested the effect of STAT-1α expression in a robustly glucose responsive INS-1 derived cell line, 832/13, which was obtained from parental, unselected INS-1 cells by stable transfection with a plasmid containing the neomycin resistance gene and harvesting of independent colonies (Hohmeier, et al. 2000). As shown in FIG. 14, incubation of 832/13 cells with 15 mM glucose stimulated insulin secretion by 12-14-fold relative to cells incubated at 3 mM glucose, regardless of whether they had first been treated with AdCMV-STAT-1α or AdCMV-βGAL or left untreated. STAT-1α overexpression has no effect on insulin secretion in a robustly glucose responsive INS-1-derived cell line.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

G. References

The following references, to the extent that they provide exemplary procedural or other details supplementary to those set forth herein, are specifically incorporated herein by reference.

U.S. Pat. No. 4,378,016

U.S. Pat. No. 5,670,488

U.S. Pat. No. 5,770,414

U.S. Pat. No. 5,792,656

U.S. Pat. No. 5,843,069

U.S. Pat. No. 5,902,745

U.S. Pat. No. 5,913,998

U.S. Pat. No. 5,980,889

U.S. Pat. No. 5,993,799

U.S. Pat. No. 5,314,471

U.S. Pat. No. 5,387,237

U.S. Pat. No. 5,824,544

U.S. Pat. No. 5,830,725

U.S. Pat. No. 5,851,826

U.S. Pat. No. 5,858,744

U.S. Pat. No. 5,879,934

U.S. Pat. No. 5,888,502

U.S. Pat. No. 5,932,210

U.S. Pat. No. 5,955,331

U.S. Pat. No. 5,994,136

U.S. Pat. No. 6,013,516

Amado and Chen, “Lentiviral vectors—the promise of gene therapy within reach?” Science, 285(5428):674-676, 1999.

Amado, Mitsuyasu, Zack, “Gene therapy for the treatment of AIDS: animal models and human clinical experience,” Front Biosci., 4:D468-475, 1999.

Asfari, M., Janjic, D., Meda, P., Li, G., Halban, P. A., Wollheim, C. B. (1992) Endocrinology 130, 167-178

Batra, Guttridge, Brenner, Dubinett, Baldwin, Boucher, “IkappaBalpha gene transfer is cytotoxic to squamous-cell lung cancer cells and sensitizes them to tumor necrosis factor-alpha-mediated cell death,” Am. J. Respir. Cell Mol. Biol., 21(2):238-245, 1999.

Bett, Prevec, Graham, “Packaging capacity and stability of human adenovirus type 5 vectors,” J. Virol., 67(10):5911-5921, 1993.

Blackwell, Miller, Douglas, Li, Peters, Carroll, Peters, Strong, Curiel, “Retargeting to EGFR enhances adenovirus infection efficiency of squamous cell carcinoma,” Arch. Otolaryngol Head Neck Surg., 125(8):856-863, 1999.

Blomer et al., J. Virol. 71:6641-6649, 1997.

Carrio, Romagosa, Mercade, Mazo, Nadal, Gomez-Foix, Fillat, “Enhanced pancreatic tumor regression by a combination of adenovirus and retrovirus-mediated delivery of the herpes simplex virus thymidine kinase gene,” Gene Ther., 6(4):547-553, 1999.

Case, Price, Jordan, Yu, Wang, Bauer, Haas, Xu, Stripecke, Naldini, Kohn, Crooks, “Stable transduction of quiescent CD34(+)CD38(−) human hematopoietic cells by HIV-1-based lentiviral vectors,″” Proc. Nat'l Acad. Sci. USA, 96(6):2988-2993, 1999.

Chillon, Bosch, Zabner, Law, Armentano, Welsh, Davidson, “Group D adenoviruses infect primary central nervous system cells more efficiently than those from group C,” J. Virol., 73(3):2537-2540, 1999.

Clay, Custer, Spiess, Nishimura, “Potential use of T cell receptor genes to modify hematopoietic stem cells for the gene therapy for cancer,” Pathol. Oncol. Res., 5(1):3-15, 1999.

Corbett and McDaniel, “Does nitric oxide mediate autoimmune destruction of β-cells? Possible therapeutic interventions in IDDM,” Diabetes, 41:897-903, 1992.

Corbett, J. A., McDaniel, M. L. (1992) Diabetes 41, 897-903Chen, G., Hohmeier, H. E., Gasa, R., Tran, V. V., and Newgard, C. B. (2000) Diabetes 49, 562-570

Derby, Sena-esteves, Breakefield, Corey, “Gene transfer into the mammalian inner ear using HSV-1 and vaccinia virus vectors,” Hear Res., 134(1-2):1-8, 1999.

Dorai, Perlman, Walsh, Shabsigh, Goluboff, Olsson, Buttyan, “A recombinant defective adenoviral agent expressing anti-bcl-2 ribozyme promotes apoptosis of bcl-2-expressing human prostate cancer cells,” Int. J. Cancer, 82(6):846-852, 1999.

Eizirik, D. L., Flodstrom, M., Karlsen, A. E., Welsh, N. (1996) Diabetologia 39, 875-890

Eizirik, D. L., M. Flodstrom, A. E. Karlsen and N. Welsh, “The harmony of the spheres; inducible nitric oxide synthase and related genes in pancreatic beta cells,” Diabetologia, 39:875-890, 1996.

Engel and Kohn, “Stem cell directed gene therapy,” Front Biosci., 4:e26-33, 1999.

Feldman, Tahlil, Steg, “Adenovirus-mediated arterial gene therapy for restenosis: problems and perspectives,” Semin Interv. Cardiol., 1(3):203-208, 1996.

Fujiwara and Tanaka, “Molecular surgery for human colorectal cancer with tumor suppressor p53 gene transfer,” Nippon Geka gakkai Zasshi, 99(7):463-468, 1998.

Garrido, Camicero, Lim, Schimmang, “Differential effects on the survival of neuronal and non-neuronal cells after infection by herpes simplex virus type 1 mutants,” J. Neurovirol., 5(30)280-288, 1999.

Gotoh et al., Transplantation 42:387, 1986.

Graham and Prevec, “Methods for construction of adenovirus vectors,” Mol. Biotechnol., 3(3):207-220, 1995.

Haddad B, Pabon-Pena C R, Young H and Sun W H, “Assignment1 of STAT1 to human chromosome 2q32 by FISH and radiation hybrids,” Cytogenet. Cell Genet. 83 (1-2), 58-59, 1998.

Haecker, Stedman, Balice-Gordon, Smith, Greelish, Mitchell, Wells, Sweeney, Wilson, “In vivo expression of full-length human dystrophin from adenoviral vectors deleted of all viral genes,″” Hum. Gene Ther., 7(15):1907-1914, 1996.

Hakan Borg et al., “Interleukin-1β increases the activity of superoxide dismutase in rat pancreatic islets,” Endocrinology, 130:2851-2857, 1992.

Halban, P. A., G. A. Praz and C. B. Wolheim, “Abnormal glucose metabolism accompanies failure of glucose to stimulate insulin release from a pancreatic islet cell line (RINm5F),” Biochem, J., 212:439-143, 1983.

Hermens and Vergaagen, “Viral vectors, tools for gene transfer in the nervous system,” Prog. Neurobiol., 55(4):399-432, 1998.

Hohmeier, et al., “Stable expression of manganese superoxide dismutase (MnSOD) in insulinoma cells prevents IL-1β-induced cytotoxicity and reduces nitric oxide production,” J. Clin. Invest., 101:1811-1820, 1998.

Hohmeier, H. E., Mulder, H., Chen, G., Prentki, M., and Newgard, C. B. (2000) Diabetes 49, 424-230

Hohmeier, H. E., Thigpen, A., Tran, V. V., Davis, R., and Newgard, C. B. (1998) J. Clin. Invest. 101, 1811-1820

Howard, Kalthoff, Fong, “Ablation of tumor cells in vivo by direct injection of HSV-thymidine kinase retroviral vector and ganciclovir therapy,” Ann. N.Y. Acad. Sci., 880-352-365, 1999.

Ilan, Saito, Thummala, Chowdhury, “Adenovirus-mediated gene therapy of liver diseases,” Semin Liver Dis., 19(1):49-59, 1999.

Irie, Anderegg, Kashani-Sabet, Ohkawa, Suzuki, Halks-Miller, Curiel, Scanlon, “Therapeutic efficacy of an adenovirus-mediated anti-H-ras ribozyme in experimental bladder cancer,” Antisense Nucleic Acid Drug Dev., 9(4):341-349, 1999.

Johnston, Gasmi, Lim, Elder, Yee, Jolly, Campbell, Davidson, Sauter, “Minimum requirements for efficient trandsduction of dividing and nondividing cells by feline immunodeficiency virus vectors,” J. Virol., 73(6):4991-5000, 1999.

Kaufmnan, et al., “Insulin pump therapy in type 1 pediatric patients: now and into the year 2000,” Diabetes Metab. Res. Rev., 15:338-52, 1999.

Kaufman, Jia, Tan, Chen, Gabelt, Booth, Tufaro, Cynader, “A perspective of gene therapy in the glaucomas,” Surv. Ophthalmol., 43(1S):91-97, 1999.

Kay, “Hepatic gene therapy for haemophilia B.,” Haemophilia, 4(4):389-392, 1998.

Klimatcheva, Rosenblatt, Planelles, “Lentiviral vectors and gene therapy,” Front Biosci., 4:D481-496, 1999.

Lachmann and Efstathiou, “Use of herpes simplex virus type 1 for transfene expression within the nervous system,” Clin. Sci., 96(6):533-541, 1999.

Larsen, et al., “Interleukin-1beta-induced rat pancreatic islet nitric oxide synthesis requires both the p38 and extracellular signal-regulated kinase ½ mitogen-activated protein kinases,” J. Biol. Chem., 273:15294-300, 1998.

Leibowitz, Beattie, Kafri, Cirulli, Lopez, Hayek, Levine, “Gene transfer to human pancreatic endocrine cells using viral vectors,” 48(4):745-753, 1999.

Lesch, “Gene transfer to the brain: emerging therapeutic strategy in psychiatry,” Biol. Psychiatry, 45(3):247-253, 1999.

Lorenz, R. “Modem insulin therapy for type 1 diabetes mellitus,” Prim. Care, 26:917-29, 1999.

Mandrup-Poulsen, T. (1996) Diabetologia 39,1005-1029

Mandrup-Poulsen, T., “The role of interleukin-1 in the pathogenesis of IDDM,” Diabetologia, 39:1005-1029, 1996.

Marienfeld, Haack, Thalheimer, Schneider-Rasp, Brackmann, Poller, “Autoreplication of the vector genome in recombinant adenorviral vectors with different E1 region deletions and transgenes, Gene Ther., 6(6):1101-1113, 1999.

Miller, Miller, Garcia, Lynch, “Use of retroviral vectors for gene transfer and expression,” Methods Enzymol., 217:581-599, 1993.

Miyake, Suzuki, Matsuoka, Tohyama, Shimada, “Stable integration of human immunodeficiency virus-based retroviral vectors into the chromosomes of nondividing cells,” Hum. Gene Ther., 9(4):467-475, 1998.

Miyatake, Tani, Feigenbaum, Sundaresan, Toda, Narumi, Kikuchi, Hashimoto, Hangai, Martuza, Rabkin, “Hepatoma-specific antitumor activity of an albumin enhancer/promoter regulated herpes simplex virus in vivo, Gene Ther., 6(4):564,5721, 1999.

Moldawer, Edwards, Josephs, Minter, Copeland, MacKay, “Application of gene therapy to acute inflammatory diseases,” Shock, 12(2):83-101, 1999.

Morrison, Onions, Nicolson, “Complete DNA sequence of canine adenovirus type 1, ” J. Gen. Virol.,78(Pt4):873-878, 1997.

Morton et al., “Interleukin-1beta activates a short STAT-3 isoform in clonal insulin-secreting cells,” FEBS Lett. 8;442(1):57-60, 1999.

Mosmann, T., “Rapid colorimetric assay for cellular growth and survival: application to proliferation and cytotoxicity assays,” J. Immunol. Meth., 65:55-63, 1983.

Naldini et al., Proc. Nat'l Acad. Sci., 93:11382-11388, 1996.

Naldini et al., Science, 272:263-267, 1996.

Newgard, C. B., S. Clark, IL. BeltrandelRio, H. E. Homeier, C. Quaade and K. Normington, “Engineered cell lines for insulin replacement in diabetes; current status and future prospects,” Diabetologia, 40:542-547, 1997.

Otonkoski, et al., “Differentiation and maturation of porcine fetal islet cells in vitro and after transplantation,” Transplantation, 68:1674-83, 1999.

Petrof, “Respiratory muscles as a target for adenovirus-mediated gene therapy,” Eur. Respir. J., 11(2):492-497, 1998.

Rabinovitch, A. (1993) Diabetes Rev. 1, 215-240.

Rabinovitch, A., “Roles of cytokines in IDDM pathogenesis and islet β-cell destruction,” Diabetes Rev. 1:215-240, 1993.

Rabinovitch, A., Suarez-Pinzon, W., Strynadka, K., Ju, Q., Edelstein, D., Brownlee, M., Korbutt, G. S., Rajotte, R. V. (1999) Diabetes 48, 1223-1229

Rabinovitch, Suarez-Pinzon, Strynadka, Ju, Edelstein, Brownlee, Korbutt, Rajotte, “Transfection of human pancreatic islets with an anti-apoptotic gene (bc1-2) protects beta-cells from cytokine-induced destruction,” Diabetes, 48(6):1223-1229, 1999.

Reddy, Idamakanti, Zakhartchouk, Baxi, Lee, Pyne, Babiuk, Tikoo, “Nucleotide sequence, genome organization, and transcription map of bovine adenovirus type 3,” J. Virol., 72(2):1394-1402, 1998.

Robbins, Tahara, Ghivizzani, “Viral vectors for gene therapy,” Trends Biotech., 16(1):35-40, 1998

Schindler, et al., “Proteins of transcription factor ISGF-3: One gene encodes the 91-and 84-kDa ISGF-3 proteins that are activated by interferon alpha,” Proc. Nat'l. Acad. Sci., 89:7836-7839, 1992.

Schnedl et al., “Streptozotocin transport and cytotoxidicy: specific enhancement in GLUT-2 expressing cells,” Diabetes, 43:1326-1333, 1994.

Shuai, et al., “A single phosphotyrosine residue of Stat91 required for gene activation by interferon-gamma,” Science, 261:1744-6, 1993.

Shuai, et al., “Activation of transcription by IFN-gamma: tyrosine phosphorylation of a 91-kD DNA binding protein,” Science, 258:1808-12, 1992.

Shuai, K., Schindler, C., Prezioso, V. R., Darnell, J. E. (1992) Science 259, 1808-1812

Smith, “Adenovirus-mediated gene transfer to treat neurologic disease,” Arch. Neurol., 55(8):1061-1064, 1998.

Steen, D., “The current state of pancreas transplantation,” AACN Clin. Issues, 10: 164-75, 1999.

Stewart, Lassam, Quirt, Bailey, Rotstein, Krajden, Dessureault, Gallinger, Cappe, Wan, Addison, Moen, Gauldie, Graham, “Adenovector-mediated gene delivery of interleukin-2 in metastatic breast cancer and melanoma: results of a phase 1 clinical trial,” Gene Ther., 6(3):350-363, 1999.

Takahashi, Miyoshi, Verma, Gage, “Rescue from photoreceptor degeneration in the rd mouse by human immunodeficiency virus vector-mediated gene transfer,” J Virol., 73(9):7812-7816, 1999.

Vanderkwaak, Wang, Gomez-Navarro, Rancourt, Dmitriev, Krasnykh, Barnes, Siegal, Alvarez, Curiel, “An advanced generation of adenoviral vectors selectively enhances gene transfer for ovarian cancer gene therapy approaches,” Gynecol. Oncol., 74(2):227-234, 1999.

Weihl, Macdonald, Stoodley, Luders, Lin, “Gene therapy for cerebrovascular disease,” Neurosurgery, 44(2):239-252, 1999.

White, Renda, Nam, Klimatcheva, Zhu, Fisk, Halterman, Rimel, Federoff, Pandya, Rosenblatt, Planelles, “Lentivirus vectors using human and simian immunodeficiency virus elements,” J. Virol., 73(4):2832-2840, 1999.

Wilson, “Adenoviruses as gene-delivery vehicles,” N. Engl. J. Med., 334(18):1185-1187, 1996.

Wu, “Recent advances in gene therapy of GI and liver diseases,” Chung Hua Min Kuo Hsiao Erh Ko I Hsueh Hui Tsa Chih, 39(5):297-300, 1998.

Yamamoto K, Kobayashi H, Arai A, Miura O, Hirosawa S and Miyasaka N., “cDNA cloning, expression and chromosome mapping of the human STAT4 gene: both STAT4 and STAT1 genes are mapped to 2q32.2→q32.3,” Cytogenet. Cell Genet. 77 (3-4), 207-210, 1997.

Yan R, Qureshi S, Zhong Z, Wen Z and Darnell JE Jr., “The genomic structure of the STAT genes: multiple exons in coincident sites in Stat1 and Stat2,” Nucleic Acids Res. 23 (3), 459-463, 1995.

Yeung, Bocklhold, Tufaro, “Efficient infection of mature skeletal muscle with herpes simplex virus vectors by using dextran sulfate as a co-receptor,” Gene Ther., 6(9):1536-1544, 1999.

Yoon, Carroll, Chiocca, Tanabe, “Influence of p53 on herpes simplex virus type 1 vectors for cancer gene therapy,” J. Gastrointest. Surg., 3(10):34-48, 1999.

Zheng, Graham, Prevec, “Transcription units of E1a, E1b and pIX regions of bovine adenovirus type 3,” J. Gen. Virol., 80(Pt7):1735-1742, 1999.

Zhong, Z., “Stat3: a STAT family member activated by tyrosine phosphorylation in response to epidermal growth factor and interleukin-6,” Science, 264:95-98, 1994.

Zhong, Z., Wen, Z. and Damell, J., “Stat3 and Stat4: members of the family of signal transducers and activators of transcription,” Proc. Natl. Acad. Sci. U.S.A. 91, 4806-4810, 1994.

Zufferey et al., Nat. Biotech., 15:871-875, 1997.

What is claimed is:

1. A method for preventing cytokine-mediated cytotoxicity of a cell introduced into a subject comprising:
a) transferring into said cell an expression cassette comprising a polynucleotide encoding STAT-1α under the control of a promoter operable in eukaryotic cells; and
b) administering said cell to said subject,
wherein expression of STAT-1α prevents cytokine-mediated cytotoxicity of transplanted cells in said subject.

2. The method of claim 1, wherein said promoter is not a native STAT-1α promoter.

3. The method of claim 2, wherein said promoter is an inducible promoter, a tissue specific promoter or a constitutive promoter.

4. The method of claim 1, wherein said cell is IL-1β resistant or IFN-γ resistant.

5. The method of claim 1, wherein said cell is IL-1β resistant and IFN-γ resistant.

6. The method of claim 1, wherein said expression cassette is contained in a replication competent vector.

7. The method of claim 6, wherein said vector is a viral.

8. The method of claim 1, wherein administering said cells to said subject comprises injection.

9. The method of claim 1, wherein administering said cells comprises encapsulating said cells in a semipermeable device, and implanting said device into said subject.

10. A method for preventing cytokine-mediated cytotoxicity of a transplanted cell comprising administering to a transplant subject an expression cassette comprising a polynucleotide encoding STAT-1α under the control of a promoter operable in eukaryotic cells, wherein expression of STAT-1α prevents cytokine-mediated cytotoxicity of said transplanted cell in said transplant subject.

11. The method of claim 10, wherein said promoter is not a native STAT-1α promoter.

12. The method of claim 11, wherein said promoter is an inducible promoter, a tissue specific promoter or a constitutive promoter.

13. The method of claim 10, wherein said expression cassette is comprised in a replication competent vector.

14. The method of claim 13, wherein said vector is a viral vector.

15. The method of claim 10, wherein administering said expression cassette to said subject is by injection.

16. The method of claim 15, wherein administering further comprises multiple injections.

17. A pharmaceutical composition comprising a polynucleotide encoding a STAT-1α operably linked to a non-STAT-1α promoter operable in eukaryotic cells, in a pharmaceutically acceptable carrier.

18. The composition of claim 17, wherein said STAT-1α comprises the sequence of SEQ ID NO:2.

19. The composition of claim 17, wherein said polynucleotide comprises the sequence of SEQ ID NO:1.

20. The composition of claim 17, wherein said promoter is an inducible promoter, a tissue specific promoter or a constitutive promoter.

21. The composition of claim 17, wherein said polynucleotide further comprises a polyadenylation signal.

22. The composition of claim 17, wherein said polynucleotide is contained in a viral vector or a plasmid.

23. The composition of claim 22, wherein said viral vector is an adenoviral vector, a retroviral vector, a vaccinia viral vector, an adeno-associated viral vector, a polyoma viral vector or a herpesviral vector.

24. A cell comprising an expression construct comprising a polynucleotide encoding a STAT-1α polypeptide operably linked to a non-STAT-1α promoter operable in eukaryotic cells, wherein said cell secretes insulin in response to glucose.

25. The cell of claim 24, wherein said STAT-1α comprises the sequence of SEQ ID NO:2.

26. The cell of claim 24, wherein said polynucleotide comprises the sequence of SEQ ID NO: 1.

27. The cell of claim 24, wherein said polynucleotide further comprises a polyadenylation signal.

28. The cell of claim 24, wherein said promoter is an inducible promoter, a tissue specific promoter or a constitutive promoter.

29. The cell of claim 24, wherein said cell is IL-1β resistant or IFN-γ resistant.

30. The cell of claim 25, wherein said cell is IL-1β resistant and IFN-γ resistant.

31. A cell comprising an expression construct comprising a polynucleotide encoding a STAT-1α polypeptide operably linked to a non-STAT-1α promoter operable in eukaryotic cells, wherein said cell secretes GLP-1.

32. A cell comprising an expression construct comprising a polynucleotide encoding a STAT-1α polypeptide operably linked to a non-STAT-1α promoter operable in eukaryotic cells, wherein said cell secretes LCAT.

33. A polynucleotide encoding a rat STAT-1α.

34. The polynucleotide of claim 33, wherein the STAT-1α has the amino acid sequence of SEQ ID NO:2.

35. The polynucleotide of claim 34, wherein the polynucleotide sequence comprises SEQ ID NO:1.

36. An expression construct comprising a polynucleotide encoding a rat STAT-1α operably linked to a promoter active in eukaryotic cells.

37. The expression construct of claim 36, further comprising an origin of replication.

38. The expression construct of claim 36, wherein said promoter is an inducible promoter, a constitutive promoter, a tissue specific promoter, or a native STAT-1α promoter.


SEQUENCE LISTING (6 sequences)

SEQ ID NO: 1 - DNA, 4003 bp, Homo sapiens
attaaacctctcgccgagcccctccgcagactctgcgccggaaagtttcatttgctgtatgccatcctcgagagctgtctaggttaacgttcgcactctgtgtatataacctcgacagtcttggcacctaacgtgctgtgcgtagctgctcctttggttgaatccccaggcccttgttggggcacaaggtggcaggatgtctcagtggtacgaacttcagcagcttgactcaaaattcctggagcaggttcaccagctttatgatgacagttttcccatggaaatcagacagtacctggcacagtggttagaaaagcaagactgggagcacgctgccaatgatgtttcatttgccaccatccgttttcatgacctcctgtcacagctggatgatcaatatagtcgcttttctttggagaataacttcttgctacagcataacataaggaaaagcaagcgtaatcttcaggataattttcaggaagacccaatccagatgtctatgatcatttacagctgtctgaaggaagaaaggaaaattctggaaaacgcccagagatttaatcaggctcagtcggggaatattcagagcacagtgatgttagacaaacagaaagagcttgacagtaaagtcagaaatgtgaaggacaaggttatgtgtatagagcatgaaatcaagagcctggaagatttacaagatgaatatgacttcaaatgcaaaaccttgcagaacagagaacacgagaccaatggtgtggcaaagagtgatcagaaacaagaacagctgttactcaagaagatgtatttaatgcttgacaataagagaaaggaagtagttcacaaaataatagagttgctgaatgtcactgaacttacccagaatgccctgattaatgatgaactagtggagtggaagcggagacagcagagcgcctgtattggggggccgcccaatgcttgcttggatcagctgcagaactggttcactatagttgcggagagtctgcagcaagttcggcagcagcttaaaaagttggaggaattggaacagaaatacacctacgaacatgaccctatcacaaaaaacaaacaagtgttatgggaccgcaccttcagtcttttccagcagctcattcagagctcgtttgtggtggaaagacagccctgcatgccaacgcaccctcagaggccgctggtcttgaagacaggggtccagttcactgtgaagttgagactgttggtgaaattgcaagagctgaattataatttgaaagtcaaagtcttatttgataaagatgtgaatgagagaaatacagtaaaaggatttaggaagttcaacattttgggcacgcacacaaaagtgatgaacatggaggagtccaccaatggcagtctggcggctgaatttcggcacctgcaattgaaagaacagaaaaatgctggcaccagaacgaatgagggtcctctcatcgttactgaagagcttcactcccttagttttgaaacccaattgtgccagcctggtttggtaattgacctcgagacgacctctctgcccgttgtggtgatctccaacgtcagccagctcccgagcggttgggcctccatcctttggtacaacatgctggtggcggaacccaggaatctgtccttcttcctgactccaccatgtgcacgatgggctcagctttcagaagtgctgagttggcagttttcttctgtcaccaaaagaggtctcaatgtggaccagctgaacatgttgggagagaagcttcttggtcctaacgccagccccgatggtctcattccgtggacgaggttttgtaaggaaaatataaatgataaaaattttcccttctggctttggattgaaagcatcctagaactcattaaaaaacacctgctccctctctggaatgatgggtgcatcatgggcttcatcagcaaggagcgagagcgtgccctgttgaaggaccagcagccggggaccttcctgctgcggttcagtgagagctcccgggaaggggccatcacattcacatgggtggagcggtcccagaacggaggcgaacctgacttccatgcggttgaaccctacacgaagaaagaactttctgctgttactttccctgacatcattcgcaattacaaagtcatggctgctgagaatattcctgagaatcccctgaagtatctgtatccaaatattgacaaagaccatgcctttggaaagtattactccaggccaaaggaagcaccagagccaatggaacttgatggccctaaaggaactggatatatcaagactgagttgatttctgtgtctgaagttcacccttctagacttcagaccacagacaacctgctccccatgtctcctgaggagtttgacgaggtgtctcggatagtgggctctgtagaattcgacagtatgatgaacacagtatagagcatgaatttttttcatcttctctggcgacagttttccttctcatctgtgattccctcctgctactctgttccttcacatcctgtgtttctagggaaatgaaagaaaggccagcaaattcgctgcaacctgttgatagcaagtgaatttttctctaactcagaaacatcagttactctgaagggcatcatgcatcttactgaaggtaaaattgaaaggcattctctgaagagtgggtttcacaagtgaaaaacatccagatacacccaaagtatcaggacgagaatgagggtcctttgggaaaggagaagttaagcaacatctagcaaatgttatgcataaagtcagtgcccaactgttataggttgttggataaatcagtggttatttagggaactgcttgacgtaggaacggtaaatttctgtgggagaattcttacatgttttctttgctttaagtgtaactggcagttttccattggtttacctgtgaaatagttcaaagccaagtttatatacaattatatcagtcctctttcaaaggtagccatcatggatctggtagggggaaaatgtgtattttattacatctttcacattggctatttaaagacaaagacaaattctgtttcttgagaagagaatattagctttactgtttgttatggcttaatgacactagctaatatcaatagaaggatgtacatttccaaattcacaagttgtgtttgatatccaaagctgaatacattctgctttcatcttggtcacatacaattatttttacagttctcccaagggagttaggctattcacaaccactcattcaaaagttgaaattaaccatagatgtagataaactcagaaatttaattcatgtttcttaaatgggctactttgtcctttttgttattagggtggtatttagtctattagccacaaaattgggaaaggagtagaaaaagcagtaactgacaacttgaataatacaccagagataatatgagaatcagatcatttcaaaactcatttcctatgtaactgcattgagaactgcatatgtttcgctgatatatgtgtttttcacatttgcgaatggttccattctctctcctgtactttttccagacacttttttgagtggatgatgtttcgtgaagtatactgtatttttacctttttccttccttatcactgacacaaaaagtagattaagagatgggtttgacaaggttcttcccttttacatactgctgtctatgtggctgtatcttgtttttccactactgctaccacaactatattatcatgcaaatgctgtattcttctttggtggagataaagatttcttgagttttgttttaaaattaaagctaaagtatctgtattgcattaaatataatatcgacacagtgctttccgtggcactgcatacaatctgaggcctcctctctcagtttttatatagatggcgagaacctaagtttcagttgattttacaattgaaatgactaaaaaacaaagaagacaacattaaaaacaatattgtttcta

SEQ ID NO: 2 - protein, 750 aa, Homo sapiens
MSQWYELQQLDSKFLEQVHQLYDDSFPMEIRQYLAQWLEKQDWEHAANDVSFATIRFHDLLSQLDDQYSRFSLENNFLLQHNIRKSKRNLQDNFQEDPIQMSMIIYSCLKEERKILENAQRFNQAQSGNIQSTVMLDKQKELDSKVRNVKDKVMCIEHEIKSLEDLQDEYDFKCKTLQNREHETNGVAKSDQKQEQLLLKKMYLMLDNKRKEVVHKIIELLNVTELTQNALINDELVEWKRRQQSACIGGPPNACLDQLQNWFTIVAESLQQVRQQLKKLEELEQKYTYEHDPITKNKQVLWDRTFSLFQQLIQSSFVVERQPCMPTHPQRPLVLKTGVQFTVKLRLLVKLQELNYNLKVKVLFDKDVNERNTVKGFRKFNILGTHTKVMNMEESTNGSLAAEFRHLQLKEQKNAGTRTNEGPLIVTEELHSLSFETQLCQPGLVIDLETTSLPVVVISNVSQLPSGWASILWYNMLVAEPRNLSFFLTPPCARWAQLSEVLSWQFSSVTKRGLNVDQLNMLGEKLLGPNASPDGLIPWTRFCKENINDKNFPFWLWIESILELIKKHLLPLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDGPKGTGYIKTELISVSEVHPSRLQTTDNLLPMSPEEFDEVSRIVGSVEFDSMMNTV

SEQ ID NO: 3 - DNA, 2260 bp, Rattus norvegicus
atgtcacagtggttcgagcttcagcagctggactccaagttcctggagcaggtgcaccagctgtacgatgacagtttccccatggaaatcagacagtacctggcccagtggctagaaaagcaagactgggagcatgctgcctacgacgtctcgttcgccaccatccgcttccatgacctgctctcccagctggatgaccagtacagccgcttttctttggagaataatttcttgttgcagcacaacataaggaaaagcaagcgtaatctccaggataacttccaagaagatcccgtacagatgtccatgattatccacaactgtctgaaggaggagaggaagattttggaaaacgcccagcgatttaatcaggcccaggagggaaatattcagaacaccgtgatgttagataaacagaaggagctggacagtaaagtcagaaatgtgaaggaccaagtcatgtgcatagagcatgaaatcaagaccctagaagacttacaagatgaatatgactttaaatgcaaaacctctcagaacagagaaagcgaagccaatggtgtggcgaagagcgaccagaaacaggaacagctgctgctgcataagatgttcttaatgcttgacaacaagagaaaggagataattcacaaaatcagagagctgctgaactccatcgagctcactcagaacactctgattaatgatgagcttgtggagtggaagcgaagacagcagagtgcctgcattgggggaccgcccaatgcctgcctggatcagctgcaaagctggttcaccattgttgcagagagcctgcagcaggtccgtcagcagcttaaaaagctggaggagttggaacagaaattcacctatgagcaggaccctattacaaaaaacaagcaggtgttatccgatcgcaccttcgtcctcttccagcagctcattcagagctcctttgtggtggaacgacagccgtgcatgccaactcaccctcagaggcccctggtcttgaagactggggtacagttcactgtgaagctgagactgttggtgaaattgcaagaactgaactataacttgaaagtgaaagtctcatttgacaaagatgtgaacgagaaaaacacagtgaaaggattccggaagttcaacatcttgggtactcacacaaaagtgatgaacatggaagaatccaccaacggaagtttggcagctgagttccgacacctgcaactgaaggaacagaaaaatgctgggaacagaaccaatgagggtcctctcattgtcaccgaagaacttcactctcttagctttgaaacccagttgtgccagccaggcttggtgattgaccttgagaccacctctcttcccgtcgtggtgatctccaatgtcagccagctccccagtggctgggcttctatcctgtggtacaacatgctggtgacagagcccaggaatctctccttcttcctgaaccccccgtgtgcgtggtggtcccagctctcagaggtgctgagttggcagttttcgtcagtcaccaagagaggtctcaacgctgaccagctgagcatgctgggggagaagcttctgggccctaatgctggccctgatggtcttattccatggacaaggttctgtaaggaaaatattaacgataagaatttctccttctggccttggattgacaccatcctagaactcattaagaagcacctgctttgcctctggaatgatgggtgcatcatgggctttatcagcaaggagcgagaacgtgctctgctcaaggaccagcagcccgggacgttcctgcttaggttcagtgagagctcccgggaaggggccatcacattcacatgggtggaacggtcccaaaatggaggtgaacccgacttccatgctgttgagccctacacgaagaaagaactttcagctgttactttcccagatattattcgcaattacaaagtcatggctgctgagaatataccagagaatcccctgaagtatctgtatcccaatattgacaaagaccatgccttcgggaagtactactccaggccaaaggaagcaccagaaccgatggagctcgatgaccctaagagaactggatacatcaagactgagttgatttctgtgtctgaagtccacccttctagacttcagagcacagaaaacctgcttcccatgtctccagaggagtttgacgagatgtccaagatagtgggctctgaatttgacagtatgatgagcgcagtataaacacgaattt

SEQ ID NO: 4 - protein, 749 aa, Rattus norvegicus
MSQWFELQQLDSKFLEQVHQLYDDSFPMEIRQYLAQWLEKQDWEHAAYDVSFATIRFHDLLSQLDDQYSRFSLENNFLLQHNIRKSKRNLQDNFQEDPVQMSMIIHNCLKEERKILENAQRFNQAQEGNIQNTVMLDKQKELDSKVRNVKDQVMCIEHEIKTLEDLQDEYDFKCKTSQNRESEANGVAKSDQKQEQLLLHKMFLMLDNKRKEIIHKIRELLNSIELTQNTLINDELVEWKRRQQSACIGGPPNACLDQLQSWFTIVAESLQQVRQQLKKLEELEQKFTYEQDPITKNKQVLSDRTFVLFQQLIQSSFVVERQPCMPTHPQRPLVLKTGVQFTVKLRLLVKLQELNYNLKVKVSFDKDVNEKNTVKGFRKFNILGTHTKVMNMEESTNGSLAAEFRHLQLKEQKNAGNRTNEGPLIVTEELHSLSFETQLCQPGLVIDLETTSLPVVVISNVSQLPSGWASILWYNMLVTEPRNLSFFLNPPCAWWSQLSEVLSWQFSSVTKRGLNADQLSMLGEKLLGPNAGPDGLIPWTRFCKENINDKNFSFWPWIDTILELIKKHLLCLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDDPKRTGYIKTELISVSEVHPSRLQSTENLLPMSPEEFDEMSKIVGSEFDSMMSAV

SEQ ID NO: 5 - DNA, 2277 bp, Mus musculus
caggatgtcacagtggttcgagcttcagcagctggactccaagttcctggagcaggtccaccagctgtacgatgacagtttccccatggaaatcagacagtacctggcccagtggctggaaaagcaagactgggagcacgctgcctatgatgtctcgtttgcgaccatccgcttccatgacctcctctcacagctggacgaccagtacagccgcttttctctggagaataatttcttgttgcagcacaacatacggaaaagcaagcgtaatctccaggataacttccaagaagatcccgtacagatgtccatgatcatctacaactgtctgaaggaagaaaggaagattttggaaaatgcccaaagatttaatcaggcccaggagggaaatattcagaacactgtgatgttagataaacagaaggagctggacagtaaagtcagaaatgtgaaggatcaagtcatgtgcatagagcaggaaatcaagaccctagaagaattacaagatgaatatgactttaaatgcaaaacctctcagaacagagaaggtgaagccaatggtgtggcgaagagcgaccaaaaacaggaacagctgctgctccacaagatgtttttaatgcttgacaataagagaaaggagataattcacaaaatcagagagttgctgaattccatcgagctcactcagaacactctgattaatgacgagctcgtggagtggaagcgaaggcagcagagcgcctgcatcgggggaccgcccaacgcctgcctggatcagctgcaaacgtggttcaccattgttgcagagaccctgcagcagatccgtcagcagcttaaaaagctggaggagttggaacagaaattcacctatgagcccgaccctattacaaaaaacaagcaggtgttgtcagatcgaaccttcctcctcttccagcagctcattcagagctccttcgtggtagaacgacagccgtgcatgcccactcacccgcagaggcccctggtcttgaagactggggtacagttcactgtcaagtcgagactgttggtgaaattgcaagagtcgaatctattaacgaaagtgaaatgtcactttgacaaagatgtgaacgagaaaaacacagttaaaggatttcggaagttcaacatcttgggtacgcacacaaaagtgatgaacatggaagaatccaccaacggaagtctggcagctgagctccgacacctgcaactgaaggaacagaaaaacgctgggaacagaactaatgaggggcctctcattgtcaccgaagaacttcactctcttagctttgaaacccagttgtgccagccaggcttggtgattgacctggagaccacctctcttcctgtcgtggtgatctccaacgtcagccagctccccagtggctgggcgtctatcctgtggtacaacatgctggtgacagagcccaggaatctctccttcttcctgaaccccccgtgcgcgtggtggtcccagctctcagaggtgttgagttggcagttttcatcagtcaccaagagaggtctgaacgcagaccagctgagcatgctgggagagaagctgctgggccctaatgctggccctgatggtcttattccatggacaaggttttgtaaggaaaatattaatgataaaaatttctccttctggccttggattgacaccatcctagagctcattaagaacgacctgctgtgcctctggaatgatgggtgcattatgggcttcatcagcaaggagcgagaacgcgctctgctcaaggaccagcagccagggacgttcctgcttagattcagtgagagctcccgggaaggggccatcacattcacatgggtggaacggtcccagaacggaggtgaacctgacttccatgccgtggagccctacacgaaaaaagaactttcagctgttactttcccagatattattcgcaactacaaagtcatggctgccgagaacataccagagaatcccctgaagtatctgtaccccaatattgacaaagaccacgcctttgggaagtattattccagaccaaaggaagcaccagaaccgatggagcttgacgaccctaagcgaactggatacatcaagactgagttgatttctgtgtctgaagtccacccttctagacttcagaccacagacaacctgcttcccatgtctccagaggagtttgatgagatgtcccggatagtgggccccgaatttgacagtatgatgagcacagtataaacacgaatttctctctggcgaca

SEQ ID NO: 6 - protein, 749 aa, Mus musculus
MSQWFELQQLDSKFLEQVHQLYDDSFPMEIRQYLAQWLEKQDWEHAAYDVSFATIRFHDLLSQLDDQYSRFSLENNFLLQHNIRKSKRNLQDNFQEDPVQMSMIIYNCLKEERKILENAQRFNQAQEGNIQNTVMLDKQKELDSKVRNVKDQVMCIEQEIKTLEELQDEYDFKCKTSQNREGEANGVAKSDQKQEQLLLHKMFLMLDNKRKEIIHKIRELLNSIELTQNTLINDELVEWKRRQQSACIGGPPNACLDQLQTWFTIVAETLQQIRQQLKKLEELEQKFTYEPDPITKNKQVLSDRTFLLFQQLIQSSFVVERQPCMPTHPQRPLVLKTGVQFTVKSRLLVKLQESNLLTKVKCHFDKDVNEKNTVKGFRKFNILGTHTKVMNMEESTNGSLAAELRHLQLKEQKNAGNRTNEGPLIVTEELHSLSFETQLCQPGLVIDLETTSLPVVVISNVSQLPSGWASILWYNMLVTEPRNLSFFLNPPCAWWSQLSEVLSWQFSSVTKRGLNADQLSMLGEKLLGPNAGPDGLIPWTRFCKENINDKNFSFWPWIDTILELIKNDLLCLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDDPKRTGYIKTELISVSEVHPSRLQTTDNLLPMSPEEFDEMSRIVGPEFDSMMSTV